SEQUENCE LISTING (20 sequences)

SEQ ID NO: 1 - protein, 34 aa, Homo sapiens
DVSTPPTVLPDNFPRYPVGKFFQYDTWKQSTQRL

SEQ ID NO: 2 - protein, 34 aa, Rattus norvegicus
DVSTSQAVLPDDFPRYPVGKFFKFDTWRQSAGRL

SEQ ID NO: 3 - protein, 34 aa, Mus musculus
DVSTSQAVLPDDFPRYPVGKFFQYDTWRQSAGRL

SEQ ID NO: 4 - protein, 34 aa, Artificial Sequence
  Description of Artificial Sequence Preptin formula sequence
DVSTXXXVLPDXFPRYPVGKFFXXDTWXQSXXRL

SEQ ID NO: 5 - protein, 33 aa, Artificial Sequence
  Description of Artificial Sequence Synthetic preptin peptide fragment
DVSTXXXVLPDXFPRYPVGKFFXXDTWXQSXXR

SEQ ID NO: 6 - protein, 32 aa, Artificial Sequence
  Description of Artificial Sequence Synthetic preptin peptide fragment
DVSTXXXVLPDXFPRYPVGKFFXXDTWXQSXX

SEQ ID NO: 7 - protein, 31 aa, Artificial Sequence
  Description of Artificial Sequence Synthetic preptin peptide fragment
DVSTXXXVLPDXFPRYPVGKFFXXDTWXQSX

SEQ ID NO: 8 - protein, 30 aa, Artificial Sequence
  Description of Artificial Sequence Synthetic preptin peptide fragment
DVSTXXXVLPDXFPRYPVGKFFXXDTWXQS

SEQ ID NO: 9 - protein, 29 aa, Artificial Sequence
  Description of Artificial Sequence Synthetic preptin peptide fragment
DVSTXXXVLPDXFPRYPVGKFFXXDTWXQ

SEQ ID NO: 10 - protein, 28 aa, Artificial Sequence
  Description of Artificial Sequence Synthetic preptin peptide fragment
DVSTXXXVLPDXFPRYPVGKFFXXDTWX

SEQ ID NO: 11 - protein, 27 aa, Artificial Sequence
  Description of Artificial Sequence Synthetic preptin peptide fragment
DVSTXXXVLPDXFPRYPVGKFFXXDTW

SEQ ID NO: 12 - protein, 33 aa, Artificial Sequence
  Description of Artificial Sequence Synthetic preptin peptide fragment
VSTXXXVLPDXFPRYPVGKFFXXDTWXQSXXRL

SEQ ID NO: 13 - protein, 32 aa, Artificial Sequence
  Description of Artificial Sequence Synthetic preptin peptide fragment
STXXXVLPDXFPRYPVGKFFXXDTWXQSXXRL

SEQ ID NO: 14 - protein, 31 aa, Artificial Sequence
  Description of Artificial Sequence Synthetic preptin peptide fragment
TXXXVLPDXFPRYPVGKFFXXDTWXQSXXRL

SEQ ID NO: 15 - protein, 30 aa, Artificial Sequence
  Description of Artificial Sequence Synthetic preptin peptide fragment
XXXVLPDXFPRYPVGKFFXXDTWXQSXXRL

SEQ ID NO: 16 - protein, 29 aa, Artificial Sequence
  Description of Artificial Sequence Synthetic preptin peptide fragment
XXVLPDXFPRYPVGKFFXXDTWXQSXXRL

SEQ ID NO: 17 - protein, 28 aa, Artificial Sequence
  Description of Artificial Sequence Synthetic preptin peptide fragment
XVLPDXFPRYPVGKFFXXDTWXQSXXRL

SEQ ID NO: 18 - protein, 27 aa, Artificial Sequence
  Description of Artificial Sequence Synthetic preptin peptide fragment
VLPDXFPRYPVGKFFXXDTWXQSXXRL

SEQ ID NO: 19 - protein, 32 aa, Artificial Sequence
  Description of Artificial Sequence Synthetic preptin peptide fragment
VSTXXXVLPDXFPRYPVGKFFXXDTWXQSXXR

SEQ ID NO: 20 - protein, 30 aa, Artificial Sequence
  Description of Artificial Sequence Synthetic preptin peptide fragment
STXXXVLPDXFPRYPVGKFFXXDTWXQSXX

(19) United States
(12) Patent Application Publication
(10) Pub. No.: US 2004/0142393 A1
(43) Pub. Date: Jul. 22, 2004

(54) Methods of use of compounds with preptin function

(76) Inventors: Garth James Smith Cooper, Auckland (NZ); Christina Maree Buchanan, Auckland (NZ); Gabriel Christopher James, Auckland (NZ)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/632,366

(22) Filed: Jul. 31, 2003

Related U.S. Application Data
(60) Provisional application No. 60/400,445, filed on Aug. 1, 2002.

(30) Foreign Application Priority Data
Aug. 1, 2002 (NZ) 520536

(51) Int. Cl. G01N 33/567; C07K 14/47

(52) U.S. Cl. 435/7.21; 530/324

(57) ABSTRACT

The invention features methods for treating various diseases, disorders and/or conditions, including injuries and wounds, as well as diseases, disorders and/or conditions for example that relate to or a recharacterized, in whole or inpart, by decreased β-cell mass, decreased β-cell number, and/or decreased β-cell function, in a subjects including humans and non-human animals. The methods include administering to a subject an effective amount of one or more compounds including preptins, preptin analogs, preptin agonists, salts thereof, and derivatives thereof

### CROSS-REFERENCE TO RELATED APPLICATIONS

This application claims the priority benefit of U.S. Patent Application Serial No. 60/400,445, filed on 1 Aug., 2002, and New Zealand Patent Application Serial No. 520536, filed on 1 Aug. 2002, both of which are herein incorporated by reference in their entirety.

### FIELD OF THE INVENTION

The subject invention pertains to compounds that are useful as mesenchymal-derived cell proliferation, enhancement, and differentiation agents and to compositions containing such compounds as active ingredients. More particularly, the invention concerns biologically active compounds, including preptins, preptin analogs, and preptin agonists, and their salts and derivatives, novel uses of the compounds, pharmaceutical compositions containing these compounds, and methods of using the compounds. Novel uses of the compounds relate to their ability to stimulate proliferation and differentiation of cells, including fibroblasts and pancreatic beta cells and cell precursors thereof, and include their uses in various diseases, disorders and conditions, including for the treatment of people with diabetes and for internal and external wound healing applications.

### BACKGROUND

Pancreatic islet β-cells play a major role in physiology, including through the secretion of insulin, a peptide hormone that exerts profound effects on intermediary metabolism. Diabetes mellitus is characterized by hyperglycemia and altered β-cell function. Type 1 diabetes is characterized by an early loss of endocrine function in the pancreas due to autoimmune destruction of the pancreatic islet β-cells, resulting in hypoinsulinemia and hyperglycemia. Type 2 diabetes is a polygenic and heterogeneous disease resulting from an interaction between genetic factors and environmental influences. See, e.g., Kecha-Kamoun et al. (2001) Diabetes Metab Res Rev, 17:146-152. Although type 2 diabetes is initially characterized by hyperinsulinemia, levels of insulin eventually decrease as a result of the loss of β-cell function and eventual β-cell failure. Progression from normal glucose tolerance, to impaired glucose tolerance, to type 2 diabetes, and to late stage type 2 diabetes is associated with altered βcell function, β-cell loss and, eventually, a decline in insulin secretion. See, e.g., Dickson et al. (2001) J. Biol. Chem., 276:21110-21120.

The pancreatic islet β-cell is at the center of diabetes research. It is the glucose responsive cell that secretes insulin to satisfy the metabolic demand of other tissues. Peripheral insulin resistance and resulting hyperglycemia characterize type 2 diabetes. β-cells often compensate for this insulin resistance with both an increase in insulin secretory capacity and β-cell mass. However, hyperglycemia worsens as β-cells fail to sustain levels of insulin output sufficient to overcome increasing resistance to insulin (Kaytor et al. (2001) J Biol Chem. 16:16). Eventual β-cell failure is primarily a failure in function but later proceeds to β-cell loss such as that seen in type 1 diabetes.

One of the most striking functional β-cell defects is a loss of acute glucose-induced insulin secretion (GIIS). β-cells initially adapt to increased demand for insulin but then decompensate as type 2 diabetes worsens. One hypothesis is that β-cells can become de-differentiated, leading to a loss of specialized functions, such as GIIS. De-differentiation of β-cells has been reported in a rat model of partial pancreatectomy that included a reduction of insulin gene expression (Weir et al. (2001) Diabetes, 50 Supplement 1, S154-S159).

Integrated networks of signaling events act in concert to control β-cell mass adaptation to insulin demand. There is some evidence to suggest that increased β-cell growth might in some part be due to a circulating growth factor. See, e.g., Flier et al. (2001) Proc. Nat. Acad. Sci. USA, 98:7475-7480, Which reported that transplantation of normal islets into the pancreas or kidney capsule of insulin resistant mice led to a marked increase in β-cell mass.

Identification of relevant growth factors, and use for stimulation of endogenous growth pathways, would be useful for increasing, for example, β-cell mass. Efforts to increase β-cell mass have been reported in several animal models of diabetes. See, e.g., Efrat, S. (2001) Diabetes, 50 Supplement 1: S189-S190; and Tourrel et al. (2002) Diabetes, 51:1443-1452.

Insulin-like growth factor 2 (IGF-II) is said to play an important role during fetal life in cell growth and differentiation of the pancreas. IGF-II is localized in the mammalian fetal and adult pancreas, and is expressed in pancreatic islet ductal epithelium, a β-cell precursor (Ilieva et al. (1999) Pancreas, 19:297-303). In the adult human pancreas, IGF-II immunoreactivity is found in the β- and ductal cells only (Portela-Gomes et al. (2000) Journal of Endocrinology, 165: 245-251). Preptin, a 34-amino acid peptide reported to correspond to Asp⁶⁹-Leu¹⁰² of the proinsulin-like growth factor II (pro-IGF-II), is present in pancreatic islet beta cells and undergoes glucose-mediated co-secretion with insulin. See Cooper and Buchanan, “Peptide Having Preptin Functionality”, WO 00/78805 (PCT/NZ00/00102). Preptin has been reported to enhance, but not initiate, insulin secretion. See Buchanan et al. (2001) Biochem. J. 360: 431-439.

### SUMMARY OF THE INVENTION

The present invention is based, in part, on discovery of the ability of preptin and other peptides to, for example, stimulate proliferation of fibroblasts and pancreatic islet β-cells. It is also based on the determination that such compounds will be useful based on their ability to promote the differentiation of other cells, for example, fibroblast and pancreatic islet β-cell precursor cells, to fibroblasts and pancreatic islet β-cells.

In one aspect, this invention features a method for treating a condition of decreased β-cell mass, decreased β-cell number, and/or decreased β-cell function, in a subject. The method includes administering an effective amount of one or more of a preptin, a preptin analog, or a preptin agonist, or salts or derivatives of the above, to the subject. As used herein, the β-cell includes pancreatic islet β-cells.

The subject can be suffering, for example, from a disease associated with partial β-cell loss, for example, β-cell loss less than about an 80% loss of β-cells. An example of such a disease includes, but is not limited to, type 2 diabetes mellitus. The subject can also be suffering from, for example, a disease associated with more substantial or complete β-cell loss, which may be characterized, for example, by greater than about an 80% loss of β-cells. An example of such a disease includes, but is not limited to, for example, type 1 diabetes mellitus and late stage type 2 diabetes.

As used herein, “preptin” is an isolated, pure or purified, or substantially pure, peptide of 34 amino acids in length, the sequence of which includes that set out in the below Formula (I), and analogs thereof:

Formula (I)

```
1 5 10
Asp Val Ser Thr R₁ R₂ R₃ Val Leu Pro Asp R₄ Phe
        15 20 25
Pro Arg Tyr Pro Val Gly Lys Phe Phe R₅ R₆ Asp
                              30
Thr Trp R₇ Gln Ser R₈ R₉ Arg Leu
```

wherein R1 is Ser or Pro, or a conservative variant of either; R₂ is Gin, or Pro or a conservative variant of either; R₃ is Ala or Thr, or a conservative variant of either; R4 is Asp or Asn, or a conservative variant of either; R₅ is Gln or Lys, or a conservative variant of either; R₆ is Tyr or Phe, or a conservative variant of either; R₇ is Arg or Lys, or a conservative variant of either; R₈ is Ala or Thr, or a conservative variant of either; and R₉ is Gly or Gin, or a conservative variant of either.

Preptin includes a human preptin or preptins, a rat preptin or preptins, and a mouse preptin or preptins, as well as allelic and species variants of each of them. Exemplary sequences of a human, a rat, and a mouse preptin are shown below:

| Human preptin: | (SEQ ID NO: 1) |
|---|---|
| 1 5 10 15 20 | |
| Asp Val Ser Thr Pro Pro Thr Val Leu Pro Asp Asn Phe Pro Arg Tyr Pro Val Gly Lys | |
| 25 30 | |
| Phe Phe Gln Tyr Asp Thr Trp Lys Gln Ser Thr Gln Arg Leu | |
| Rat preptin: | (SEQ ID NO: 2) |
| 1 5 10 15 20 | |
| Asp Val Ser Thr Ser Gln Ala Val Leu Pro Asp Asp Phe Pro Arg Tyr Pro Val Gly Lys | |
| 25 30 | |
| Phe Phe Lys Phe Asp Thr Trp Arg Gln Ser Ala Gly Arg Leu | |
| Mouse preptin: | (SEQ ID NO: 3) |

1 5 10 15 20
Asp Val Ser Thr Ser Gln Ala Val Leu Pro Asp Asp Phe Pro Arg Tyr Pro Val Gly Lys
25 30
Phe Phe Gln Tyr Asp Thr Trp Arg Gln Ser Ala Gly Arg Leu

Analogs of preptin include functional equivalents of preptin, e.g., functional equivalents of the compounds of Formula (I). In terms of preptin itself, functional equivalents include proteins that are immunologically cross-reactive with and have one or more of the functions of preptin, for example, those having substantially the same function or functions as preptin (e.g., any of SEQ ID NOs: 1-3). Various preptin analogs include, for example, include C-terminal truncations of preptin, including preptin 1-33, preptin 1-32, preptin 1-31, preptin 1 -30, preptin 1-29, preptin 1-28, and preptin 1-27. Other preptin analogs include, for example, various N-terminal truncations of preptin, including preptin 2-34, preptin 3-34, preptin 4-34, preptin 5-34, preptin 6-34, preptin 7-34, and preptin 8-34. Others preptin analogs include various fragments having both N-terminal truncations and C-terminal truncations, for example, preptin 2-33 and preptin 3-32. Other preptin analogs include peptides including a preptin active site or sites. Still other compounds within the scope of the invention include preptins and preptin analogs having one or more amino acid substitutions, preferably one or more conservative amino acid substitutions. Other preptin analogs include addition and deletion mutants of a preptin or analog thereof, as well as peptides comprising, consisting essentially of, or consisting of, a fusion of a preptin, or a fragment or a mutant thereof, with other amino acids or peptides.

“A preptin agonist” is a compound which (1) has a high or other desired affinity for a preptin-binding receptor, e.g., about a Ka from about 10⁷ to about 10⁹ M, or a Ka from about 10⁸ to about 10⁹ M, or greater (as may be measured, for example, by a receptor binding assay having a format such as that described in Motulsky and Mahan (I984). Mol. Pharmacol. 25: 1); and/or (2) like preptin, stimulates proliferation of certain cells, e.g., fibroblast cells, such as NIH-3T3 cells, or β-cells, such as INS-LE β-cells.

Agonists within the scope of the invention include compounds comprising, consisting essentially or, or consisting of, (A) a preptin or a functional fragment thereof that is attached, directly or indirectly, preferably at the N-terminus of preptin, to (B) all or a portion of the amino acid sequence corresponding to IGF-II (i.e., pro-IGF-II 1-68). These peptides, i.e., peptides (A) and (B) may be joined indirectly through one or more other amino acids, for example, an arginine residue.

Still other agonists include compounds comprising, consisting essentially or, or consisting of, (A) a preptin or a functional fragment thereof that is attached, directly or indirectly, preferably a t the C -terminus of preptin, to (C) all or a portion of the amino acid sequence corresponding to pro-IGF-II 103-156. These peptides, i.e., peptides (A) and (C) may also be joined indirectly through one or more other amino acids, for example, an arginine residue.

Still other agonists include compounds comprising, consisting essentially or, or consisting of, peptides (A) and (B) and (C), joined directly or indirectly in any combination. Thus, such peptides include, for, example, peptide (13) linked directly or indirectly to peptide (A), which in turn is linked directly or indirectly to peptide (C); peptide (A) linked directly or indirectly to peptide (B), which in turn is linked directly or indirectly to peptide (C); peptide. (B) linked directly or indirectly to peptide (C), which in turn is linked directly or indirectly to peptide (A); and so on.

Still other agonists include fragments of pro-IGF-II that include all of preptin, i.e., all 34 amino acids in sequence.

Additionally, for example, a preptin agonist may be a peptide comprising, consisting essentially or, or consisting of, less than about 87 amino acids or more than 20 amino acids, and containing, in consecutive sequence, any part or all, preferably all, of SEQ ID NO: 1, 2, or 3. In another example, a preptin agonist may be a pep tide comprising, consisting essentially or, or consisting of, less than about 87 amino acids or more than 35 amino acids, and containing, in consecutive sequence, any part or all, preferably all, of the amino acid sequence of SEQ ID NO: 1, 2, or 3.

In one embodiment, the methods described herein include administering to a subject an effective amount of a preptin comprising, consisting essentially or, or consisting of, the amino acid sequence of any of Formula (I) or SEQ ID NOs: 1, 2, or 3.

In one embodiment, the methods described herein include administering to a subject an effective amount of a preptin that is an allelic or species variant of any of the peptides of Formula (I) or SEQ ID NOs: 1, 2, or 3.

In another embodiment, the methods described herein include administering to a subject an effective amount of a preptin analog, including, for example, an analog of a preptin comprising, consisting essentially or, or consisting of, the amino acid sequence of any of Formula (I) or SEQ ID NOs: 1, 2, or 3.

In another embodiment, the method includes administering to a subject an effective amount of a preptin agonist. Such embodiments include administering to a subject an effective amount of a preptin agonist containing an amino acid sequence that is at least about 60% (e.g., at least about 70%, at least about 80%, at least about 90%, at least about 95% to at least about 98%, or at least about 99%) identical to Formula (I) or to SEQ ID NO: 1, 2, or 3.

In still another embodiment, the method includes administering to a subject an effective amount of a preptin agonist containing SEQ ID NO: 1, 2, or 3 with up to fourteen conservative amino acid substitutions.

In another aspect, this invention features a method for increasing or maintaining β-cell mass. The method includes administering to a subject in need thereof an effective amount of preptin, a preptin analog, or a preptin agonist as described herein. As used herein, the subject may have a substantially normal β-cell mass, increased β-cell mass, reduced β-cell mass or the subject may be at risk of β-cell loss or dysfunction. In a further aspect, this invention features a method for stimulating β-cell growth, or increasing β-cell number. Without wishing to be bound by any particular theory or mechanism(s) of action, such stimulation or increase may be, for example, via cell differentiation or neogenesis. The method includes administering to a subject in need thereof an effective amount of one or more of a preptin, a preptin analog, or a preptin agonist, or one or more salts or derivatives of any of them.

In another aspect, the invention relates to a method of treating any disease, disorder or condition mediated in whole or in part by β-cells, any disease, disorder or condition involving β-cells, in whole or in part, and any disease, disorder or condition characterized in whole or in part by β-cell dysfunction, or any disease, disorder or condition that would be benefited by enhanced β-cell function, in a subject comprising administering to the subject an effective amount of one or more of a preptin, a preptin analog, or a preptin agonist, or one or more salts or derivatives of any of them. Preptins include the amino acid sequence of any of Formula (I), and SEQ ID NO: 1, 2, or 3. Preptin agonists include, for example, a fragment or the entirety of the amino acid sequence of any of SEQ ID NO: 1, 2, or 3.

Another aspect is a method of increasing insulin secretion in a subject comprising administering to the subject an effective amount of one or more of a preptin, a preptin analog, or a preptin agonist, or one or more salts or derivatives of any of them. The subject, for example, is one who has decreased insulin production or function (e.g., insulin resistance or reduced β-cell mass, or reduced β-cell function, or, for example diagnosed with diabetes). In one aspect, the method involves increasing insulin secretion by or in the subject's β-cells, or increasing their β-cell mass, or increasing their number.

In other aspects, the methods delineated herein include determining that a subject is in need of modulation of (e.g., increasing, maintaining, etc.) β-cell mass, β-cell number, β-cell growth, or β-cell function Determining subject status can be subjective (e.g., opinion of subject or a health care provider, or based on observation or other general symptom or parameter) or can be objective (e.g., measurable by a test or diagnostic marker) by direct or indirect analysis or evaluation or prognostication of cell mass, cell function, or cell number.

In another aspect, the invention provides a method for treating loss of β-cell mass, number or function with an effective amount of a one or more of a preptin, a preptin analog, or a preptin agonist, or one or more salts or derivatives of any of them.

In another aspect, the invention provides a method for increasing or maintaining β-cell mass, number or function with a one or more of a preptin, a preptin analog, or a preptin agonist, or one or more salts or derivatives of any of them.

This invention also features an article of manufacture that includes a vessel containing one or more of a preptin, a preptin analog, or a preptin agonist, or one or more salts or derivatives of any of them, and instructions for use of the contents for the treatment of a disease involving, for example, decreased β-cell mass, number or function (relative to normal β-cell mass, number or function, or other diagnosis indicating below normal levels of β-cell mass, number or function) comprising administering an effective amount of one or more of a preptin, a preptin analog, or a preptin agonist, or one or more salts or derivatives of any of them to a subject.

Also within the scope of this invention is an article of manufacture that includes packaging material; and contained within the packaging material, one or more of a preptin, a preptin analog, or a preptin agonist, or one or more salts or derivatives of any of them. The packaging material comprises a label that indicates that the contents can be used for treating a condition such as, for example, a condition mediated by β-cell loss or dysfunction (e.g., type 1 or type 2 diabetes mellitus) in a subject. In other aspects, the label includes dosage information.

In another aspect the present invention is directed to the use of an effective amount of one or more of a preptin, a preptin analog, or a preptin agonist, or one or more salts or derivatives of any of them in the manufacture, with or without other material or materials (whether, for example, other active ingredients, excipients, diluents or the like, and/or whether a within dosage unit defining vessel), of a dosage unit effective for use in a method of the present invention or for any of the purposes herein described or provided.

In a further aspect, the invention also provides for the use of one or more of a preptin, a preptin analog, or a preptin agonist, or one or more salts or derivatives of any of them in the treatment of any of a wide range of injuries, including wounds. Examples of the types of wounds treatable using one or more of a preptin, a preptin analog, or a preptin agonist, or one or more salts or derivatives of any of them include, but are not limited to, chemical and thermal bums; skin graft donor and transplant sites; cutaneous ulcers, including but not limited to decubitus ulcers, diabetic ulcers, vascular stasis ulcers, and necrobiosis lipoidicum ulcers; surgical wounds, wound dehiscence, including but not limited to the abdominal, thigh, and chest areas; corneal trauma and transplants; tooth extractions and oral surgery; disruption of a mucous membrane, including but not limited to the gastrointestinal tract (for example, ulcerative colitis and Crohn's Disease) and bladder; and any of a wide range of other traumatic disruptions and interruptions of the skin or connective tissue, for example, abrasions.

Certain preferred formulations of the invention designed for topical administration, for example, include ointments, creams, and gels. A preferred topical formulation for use in accordance with this invention is an ointment.

In another aspect, the present invention provides a method for treating an injury or wound in or on a subject, which comprises applying or administering to said injury or wound, or to said subject, a composition comprising an effective amount of one or more compounds selected from the group consisting of preptins, preptin analogs, and preptin agonists, or one or more salts or derivatives of any of them. Preferably said subject is a human. Other subjects may be non-human, and include domesticated and commercial animals. Said compositions include, but are not limited to, ointments, creams, and gels.

Examples of wounds that may be treated in accordance with the invention include, but are not limited to, (1) wounds in which the skin or another external surface is torn, pierced, cut, or otherwise broken; (2) wounds in which the flesh has been penetrated but the underlying bones or vital organs are undamaged or substantially undamaged; (3) skin surface injuries. In another example, a wound comprises internal bleeding.

In another aspect the present invention provides a method for enhancing wound healing, which comprises applying, for example, topically or internally, to such wound one or more compounds selected from the group consisting of a preptin, a preptin analog, or a preptin agonist, or a salt and/or derivative of any of them.

In another aspect the present invention provides a method for treating a condition in a subject by the application or administration of a compound that promotes the proliferation of mesenchymal-derived cells and/or cell mass, the improvement comprising administering to said subject an effective amount of one or more compounds selected from the group consisting of one or more of a preptin, a preptin analog, or a preptin agonist, or one or more salts or derivatives of any of them. Mesenchymal-derived cells include, but are not limited to, fibroblasts.

In another aspect the present invention provides the administration of a composition comprising an effective amount of one or more compounds selected from the group consisting of preptins, preptin analogs, and preptin agonists for the treatment and/or prevention of any one or more of a peripheral nervous system injury, Alzheimer's disease, apoplexy, amyotrophic lateral sclerosis, Parkinson's disease and the like, muscular dystrophy, diabetic neuropathy, and myocardiopathies including myocarditis and myocardial infarction, cardiac disease, and acute attack, and acute renal insufficiency caused by ischemia.

In another aspect the present invention provides the administration of a composition comprising an effective amount of one or more compounds selected from the group consisting of preptins, preptin analogs, preptin agonists, and salts and/or derivatives of any of them, for decreasing cell death of motor neurons, increasing -muscular end plates, promoting the functional recovery of damaged sciatic nerves, preventing peripheral motor paralysis observed during chemotherapy, and improvement of myocardial function.

In another aspect the present invention provides the method for promoting the growth of tissues in a subject by the application or administration of a compound that promotes such growth, which comprises applying or administering to said subject an effective amount of one or more compounds selected from the group consisting of preptins, preptin analogs, preptin agonists, and salts and/or derivatives of any of them. Examples of preferred tissues are connective tissues and/or epithelial tissues and/or pancreatic tissue.

In another aspect the present invention provides a method for improving the immune function in a subject by the application or administration of a compound to improve immune function, which comprises applying or administering to said subject an effective amount of one or more compounds selected from the group consisting of preptins, preptin analogs, preptin agonists, and salts and/or derivatives of any of them. One example, of this aspect of the invention includes the improvement of the proliferation of lymphocytes.

In another aspect of the present invention also provides polynucleotides or vectors (including cloning vectors and expression vectors) or transformed or transfected cells, including isolated or purified or pure polynucleotides, vectors, and isolated transformed or transfected cells, encoding or containing any one of the above or herein described polypeptide or protein constructs of the invention, for example, including analogs, fragments, agonists, and fusion proteins. Thus, in various embodiments the invention provides a recombinant cloning or expression construct comprising any such polynucleotide that is operably linked to a promoter.

In other embodiments there is provided a host cell transformed or transfected with, or otherwise containing, any such recombinant cloning or expression construction Host cells include the cells of a subject undergoing ex vivo cell therapy including, for example, ex vivo gene therapy for a β-cell disorder or for bum or other wounds.

In a related embodiment there is provided a method of producing a polypeptide or protein or other construct of the invention, for example, including a preptin, a preptin analog, and/or a preptin agonist protein, comprising the steps of (a) culturing a host cell as described or provided for herein under conditions that permit expression of the construct, for example, a preptin analog or fusion protein; and (b) isolating the construct, for example, a preptin, a preptin analog, and/or a preptin agonist protein from the host cell or host cell culture.

In another embodiment the invention provides a pharmaceutical composition comprising, for example, an isolated, purified, or pure polynucleotide encoding any one of the polypeptide or protein constructs of the invention, for example (including, for example, preptin analogs, agonists, and/or fusion proteins), in combination with a physiologically acceptable carrier, or for example, in combination with, or in, a gene therapy delivery vehicle or vector.

Other features, objects, and advantages of the invention will be apparent from the description and drawings, and from the claims.

### BRIEF DESCRIPTION OF THE DRAWINGS

FIG. 1 shows the dose dependent effect of preptin on the proliferation of NIH-3 T3 fibroblast cells (measured by tritiated thymidine uptake).

FIG. 2 compares the effect of preptin, GLP-1 and IGF-II on the proliferation of INS-lE β-cell s (measured by tritiated thymidine uptake).

### DETAILED DESCRIPTION OF THE INVENTION

This invention relates to use of one or more of a preptin, a preptin analog, a preptin agonist, or a salt and/or derivative of any of them, for stimulating the proliferation or expansion of mesenchymal-derived cells and/or cell mass or function, such as fibroblasts, and for stimulating the proliferation of β-cell growth or increasing β-cell mass and/or function. Without wishing to be bound by any particular theory or mechanism(s) of action, such stimulation or increase may be, for example, via cell differentiation or neogenesis.

Preptin may be isolated from pancreatic islet beta cells in a manner according to Buchanan et al. (2001) Biochem. J. 360: 431-439, for example. Preptins, as well as preptin analogs and agonists, and salts and derivatives thereof, may also be prepared using synthetic methods. Syntheses of peptides and their salts and derivatives, including both solid phase and solution phase peptide syntheses, are well established in the art. See, e.g., Stewart, et al. (1984) Solid Phase Peptide Synthesis (2^{nd} Ed.); and Chan (2000) “Fmoc Solid Phase Peptide Synthesis, A Practical Approach,” Oxford University Press. Peptides may be synthesized using an automated peptide synthesizer (e.g., a Pioneer™ Peptide Synthesizer, Applied Biosystems, Foster City, Calif.). For example, a peptide is prepared on methylbenzyhydrylamine resin followed by hydrogen fluoride deprotection and cleavage from the resin.

Proteins and peptides useful in the invention may also be prepared by recombinant methods. The present invention provides recombinant expression constructs capable of directing the expression of proteins and peptides useful in the present invention. The amino acids, which occur in the various amino acid sequences referred to herein, are identified according to their well known three-letter or single-letter abbreviations. The nucleotides, which occur in the various DNA sequences or fragments thereof referred herein, are designated with the standard single letter designations used routinely in the art. A given amino acid sequence may also encompass similar but changed amino, acid sequences, such as those having only minor changes, for example by way of illustration and not limitation, covalent chemical modifications, insertions, deletions and substitutions, which may further include conservative substitutions or substitutions with non-naturally-occuring amino acids. Amino acid sequences that are similar to one another may share substantial regions of sequence homology. In like fashion, nucleotide sequences may encompass substantially similar nucleotide sequences having only minor changes, for example by way of illustration and not limitation, covalent chemical modifications, insertions, deletions and substitutions, which may further include silent mutations owing to degeneracy of the genetic code. Nucleotide sequences that are similar to one another may share substantial regions of sequence homology.

As used herein, an “amino acid” is a molecule having the structure wherein a central carbon atom (the alpha (α)-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino and carboxylic groups in the dehydration reaction that links one amino acid to another. As a result, when incorporated into a protein, an amino acid may also be referred to as an “amino acid residue.” In the case of naturally occurring proteins, an amino acid residue's R group differentiates the 20 amino acids from which proteins are typically synthesized, although one or more amino acid residues in a protein may be derivatized or modified following incorporation into protein in biological systems (e.g., by glycosylation and/or by the formation of cystine through the oxidation of the thiol side chains of two non-adjacent cysteine amino acid residues, resulting in a disulfide covalent bond that frequently plays an important role in stabilizing the folded conformation of a protein, etc.). As those in the art will appreciate, non-naturally occurring amino acids can also be incorporated into proteins, particularly those produced by synthetic methods, including solid state and other automated synthesis methods. Examples of such amino acids include, without limitation, α-amino isobutyric acid, 4-amino butyric acid; L-amino butyric acid, 6-amino hexanoic acid, 2-amino isobutyric acid, 3-amino propionic acid, ornithine, norlensine, -norvaline, hydroxproline, sarcosine, citralline, cysteic acid, t-butylglyine, t-butylalanine, phenylylycine, cyclohexylalanine, β-alanine, fluoro-amino acids, designer amino acids (e.g. β-methyl amino acids, α-methyl amino acids, Nα-methyl amino acids) and amino acid analogs in general. In addition, when an α-carbon atom has four different groups (as is the case with the 20 amino acids used by biological systems to synthesize proteins, except for glycine, which has two hydrogen atoms bonded to the α carbon atom), two different enantiomeric forms of each amino acid exist, designated D and L. In mammals, only L-amino acids are incorporated into naturally occurring polypeptides. The instant invention envisions proteins incorporating one or more D-and L- amino acids, as well as proteins comprised of just D- or L- amino acid residues.

Herein, the following abbreviations may be used for the following amino acids (and residues thereof): alanine (Ala, A); arginine (Arg, R); asparagine (Asn, N); aspartic acid (Asp, D); cyteine (Cys, C); glycine (Gly, G); glutamic acid (Glu, E); glutamine (Gln, Q); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V). Non-polar (hydrophobic) amino acids include alanine, leucine, isoleucine, valine, proline, phenylalanine, tryptophan, and methionines. Neutral amino acids include glycine, serine, threonine, cysteine, tyrosine, esparagine, and glutamine. Positively charged (basic amino acids include arginine, lysine and histidine. Negatively charged (acidic) amino acids include aspartic acid and glutamic acid. “Protein” or “peptide” refers to any polymer of two or more individual amino acids (whether or not naturally occurring) linked via a peptide bond, and occurs when the carboxyl carbon atom of the carboxylic acid group bonded to the α-carbon of one amino acid (or amino acid residue) becomes covalently bound to the amino nitrogen atom of amino group bonded to the a-carbon of an adjacent amino acid. The term “protein” is understood to include the terms “polypeptide” and “peptide” (which, at times, may be used interchangeably herein) within its meaning, and vice versa. In addition, proteins comprising multiple polypeptide subunits or other components will also be understood to be included within the meaning of “protein” as used herein. Similarly, fragments of proteins, peptides, and polypeptides are also within the scope of the invention and may be referred to herein as “proteins.”

In biological systems (be they in vivo or in vitro, including cell-free, systems), the particular amino acid sequence of a given protein (i.e., the polypeptide's “primary structure,” when written from the amino-terninus to carboxy-terminus) is determined by the nucleotide sequence of the coding portion of a mRNA, which is in turn specified by genetic information, typically genomic DNA (which, for purposes of this invention, is understood to include organelle DNA, for example, mitochondrial DNA and chloroplast DNA). Of course, any type of nucleic acid that constitutes the genome of a particular organism (e.g., double-stranded DNA in the case of most animals and plants, single or double-stranded RNA in the case of some viruses, etc.) is understood to code for the gene product(s) of the particular organism. Messenger RNA is translated on a ribosome, which catalyzes the polymerization of a free amino acid, the particular identity of which is specified by the particular codon (with respect to mRNA, three adjacent A, G, C, or U ribonucleotides in-the mnRNA's coding region) of the rnRNA then being translated, to a nascent polypeptide. Recombinant DNA techniques have enabled the large-scale synthesis of proteins and polypeptides (e.g., human insulin, human growth hormone, erythropoietin, granulocyte colony stimulating factor, etc.) having the same primary sequence as when produced naturally in living organisms. In addition, such technology has allowed the synthesis of analogs of these and other proteins, which analogs may contain one or more amino acid deletions, insertions, and/or substitutions as compared to the native proteins. Recombinant DNA technology also enables the synthesis of entirely novel proteins.

In non-biological systems (e.g., those employing solid state synthesis), the primary structure of a protein (which also includes disulfide (cystine) bond locations) can be determined by the user. As a result, polypeptides having a primary structure that duplicates that of a biologically produced protein can be achieved, as can analogs of such proteins. In addition, completely novel polypeptides can also be synthesized, as can protein incorporating non-naturally occurring amino acids.

The term “gene” means a segment of DNA involved in producing a polypeptide chain; it may also include regions preceding and following a polypeptide coding region, for example, a “leader and trailer” as well as intervening sequences (introns) between relevant individual coding segments (exons).

As described herein, the invention provides for peptides that may be encoded in whole or in part by nucleic acids that have a preptin coding sequence fused or otherwise connected in frame to an additional native or engineered sequences to provide for expression of a polypeptide sequence fused or otherwise connected to an additional functional polypeptide sequence that permits, for example by way of illustration and not limitation, detection, functional alteration, isolation and/or purification of the fusion protein.

Modification of a polypeptide may be effected by any means known to those of skill in this art. The preferred methods herein rely on modification of DNA encoding, for example, a preptin protein, analog or agonist, and expression of the modified DNA. DNA encoding one of the peptide constructs of the invention may be altered or mutagenized using standard methodologies, including those described below.

Conservative substitutions of amino acids are well-known and may be made generally without altering the biological activity of the resulting protein molecule. For example, such substitutions are generally made by interchanging within the groups of polar residues, charged residues, hydrophobic residues, small residues, and the like. If necessary, such substitutions may be determined empirically merely by testing the resulting modified protein for the ability to bind to the appropriate cell surface receptors in in vitro biological assays, or to bind to appropriate antigens or desired target molecules.

The present invention further relates to nucleic acids which hybridize to constructs of the invention, including for example, preptin-, preptin analog-, and/or preptin agonist-encoding polynucleotide sequences as provided herein, or their complements, as will be readily apparent to those familiar with the art, if there is at least about 70%, or at least about 80-85%, or at least about 90%, or at least about 95%, 96%, 97%, 98% or 99% identity between the sequences.

The present invention particularly relates to nucleic acids that hybridize under stringent conditions to, for example, the preptin-, preptin analog-, and/or preptin agonist-encoding nucleic acids referred to herein. As used herein, to “hybridize” under conditions of a specified stringency is used to describe the stability of hybrids formed between two single-stranded nucleic acid molecules. Stringency of hybridization is typically expressed in conditions of ionic strength and temperature at which such hybrids are annealed and washed. The term “stringent conditions” refers to conditions that permit hybridization between polynucleotides. Stringent conditions can be defined by salt concentration, the concentration of organic solvent (e.g., formamide), temperature, and other conditions well known in the art. In particular, stringency can be increased by reducing the concentration of salt, increasing the concentration of organic solvents (e.g., formamide), or raising the hybridization temperature. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of an organic solvent (e.g., at least about 35% formamide, most preferably at least about 50% formamide). Stringent temperature conditions will ordinarily include temperatures of at least about 30° C., more preferably of at least about 37° C., and most preferably of at least about 42° C. Varying additional parameters, for example, hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed, and are within the skill in the art. Other typical “high”, “medium” and “low” stringency encompass the following conditions or equivalent conditions thereto: high stringency: 0.1×SSPE or SSC, 0.1% SDS, 65° C; medium stringency: 0.2×SSPE or SSC, 0.1% SDS, 50° C; and low stringency: 1.0×SSPE or SSC, 0.1% SDS, 50° C. As known to those having ordinary skill in the art, variations in stringency of hybridization conditions may be achieved by altering the time, temperature and/or concentration of the solutions used for prehybridization, hybridization and wash steps, and suitable conditions may also depend in part on the particular nucleotide sequences of the probe used, and of the blotted, proband nucleic acid sample. Accordingly, it will be appreciated that suitably stringent conditions can be readily selected without undue experimentation where a desired selectivity of the probe is identified, based on its ability to hybridize to one or more certain proband sequences while not hybridizing to certain other proband sequences.

As used herein, preferred “stringent conditions” generally refer to hybridization that will occur only if there is at least about 90-95%, or at least about 97% identity between the sequences. The nucleic acid constructs which hybridize to, for example, preptin-, preptin analog-, and/or a preptin agonist-encoding nucleic acids referred to herein, in preferred embodiments, encode polypeptides which retain substantially the same biological function or activity as, for example, the preptin, preptin analog, and/or preptin agonist polypeptides encoded by the cDNAs.

The nucleic acids of the present invention, also referred to herein as polynucleotides, may be in the form of RNA, for example, mRNA, or in the form of DNA, which DNA includes cDNA (also called “complementary DNA”, which is a DNA molecule that is complementary to a specific messenger RNA), genomic DNA, and synthetic DNA. The DNA may be double-stranded or single-stranded, and if single stranded may be the coding strand or non-coding (anti-sense) strand. A coding sequence that encodes a preptin to be used in the invention, for example, may contain portions that are identical to the coding sequence known in the art or described herein for portions thereof, or may be a different coding sequence, which, as a result of the redundancy or degeneracy of the genetic code, encodes the same construct or portion thereof, including all or a portion of a preptin, a preptin analog, and/or a preptin agonist polypeptide.

The nucleic acids that encode constructs useful in the invention for use according to the invention may include, but are not limited to: only the coding sequence for a preptin. The term “nucleic acid encoding” or “polynucleotide encoding” a preptin, for example, encompasses a nucleic acid which includes only coding sequence for, for example, a preptin polypeptide as well as a nucleic acid which includes additional coding and/or non-coding sequence(s). Nucleic acids and oligonucleotides for use as described herein can be synthesized by any method known to those of skill in this art (see, e.g., WO 93/01286, U.S. application serial No. 07/723,454; U.S. Pat. No. 5,218,088; U.S. Pat. No. 5,175,269; U.S. Pat. No. 5,109,124). Identification of various oligonucleotides and nucleic acid sequences also involves methods known in the art. F or example, the desirable properties, lengths and other characteristics of oligonucleotides useful for cloning are well known. In certain embodiments, synthetic oligonucleotides and nucleic acid sequences may be designed that resist degradation by endogenous host cell nucleolytic enzymes by containing such linkages as: phosphorothioate, methylphosphonate, sulfone, sulfate, ketyl, phosphorodithioate, phosphoramidate, phosphate esters, and other such linkages that have proven useful in antisense applications. See, e.g., Agrwal et al., Tetrehedron Lett. 28:3539-3542 (1987); Miller et al., J. A m. Chem. Soc. 9 3:6657-6665 (1971); S tec et al., Tetrehedron Lett. 2 6:2191-2194 (1985); Moody et al., Nuc. Acids Res.12:4769-4782 (1989); Uznanski et al., Nucl. Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988).

As used herein “deletion” has its common meaning as understood by those familiar with the art, and may refer to molecules that lack one or more portions of a sequence from either terminus or from a non-terminal region, relative to a corresponding full length molecule, for example, as in the case of truncated molecules provided herein. Truncated molecules that are linear biological polymers such as nucleic acid molecules or polypeptides may have one or more of a deletion from either terminus of the molecule and/or one or more deletions from a non-terminal region of the molecule.

The present invention further relates to variants of the herein referenced nucleic acids that encode fragments, analogs and/or derivatives of a construct of the invention, for example, a preptin polypeptide. The variants of the nucleic acids encoding constructs of the invention may be naturally occurring allelic variants of one or more portions of the the nucleic acid sequences included therein, or non-naturally occurring variants of such sequences or portions or sequences, including sequences varied by molecular engineering using, for example, methods know in the art for varying sequence. As is known in the art, an allelic variant is an alternate form of a nucleic acid sequence which may have at least one of a substitution, a deletion or an addition of one or more nucleotides, any of which does not substantially or undesirably alter the function of the encoded preptin, preptin analog, and/or preptin agonist, for example.

Variants and derivatives of constructs of the invention, for example, preptin proteins, may be obtained by mutations of nucleotide sequences encoding, for example, preptin, preptin analog, and/or preptin agonist polypeptides or any portion thereof. Alterations of the native amino acid sequence may be accomplished by any of a number of conventional methods. Mutations can be introduced at particular loci, for example, by synthesizing oligonucleotides containing, a mutant sequence, flanked by restriction sites enabling ligation to fragments of the native sequence. Following ligation, the resulting reconstructed sequence encodes an analog having the desired amino acid insertion, substitution, or deletion.

Alternatively, for example, oligonucleotide-directed site-specific mutagenesis procedures can be employed to provide an altered gene wherein predetermined codons can be altered by substitution, deletion or insertion. Exemplary methods of making such alterations are disclosed by Walder et al., 1986 Gene 42:133; Bauer et al., 1985 Gene 37:73; Craik, January 1985 BioTechniques 12-19; Smith et al., January 1985 Genetic Engineering: Principles and Methods BioTechniques 12-19; Costa GL, et al., “Site-directed mutagenesis using a rapid PCR-based method,” 1996 Methods Mol Biol. 57:239-48; Rashtchian A., “Novel methods for cloning and engineering genes using the polymerase chain reaction,” 1995 Curr Opin Biotechnol. 6(1):30-6; Sharon J, et al., “Oligonucleotide-directed mutagenesis of antibody combining sites,” 1993 Int Rev Immunol. 10(2-3):113-27; Kunkel, 1985 Proc. Natl. Acad. Sci. USA 82:488; Kunkel et al., 1987 Methods in Enzymol. 154:367; and, U.S. Pat. Nos. 4,518,584 and 4,737,462.

As an example, modification of DNA may be performed by site-directed mutagenesis of DNA encoding a protein combined with the use of DNA amplification methods using primers to introduce and amplify alterations in the DNA template, such as PCR splicing by overlap extension (SOE). Site-directed mutagenesis is typically effected using a phage vector that has single- and double-stranded forms, such as M13 phage vectors, which are well-known and commercially available. Other suitable vectors that contain a single-stranded phage origin of replication may be used. See, e.g., Veira et al., 1987 Meth. Enzymol. 15:3. In general, site-directed mutagenesis is performed by preparing a single-stranded vector that encodes the protein of interest (e.g., all or a component portion of a given binding preptin, preptin analog, and/or preptin agonist protein). An oligonucleotide primer that contains the desired mutation within a region of homology to the DNA in the single-stranded vector is annealed to the vector followed by addition of a DNA polymerase, such as E. coli DNA polymerase I (Klenow fragment), which uses the double stranded region as a primer to produce a heteroduplex in which one strand encodes the altered sequence and the other the original sequence. The heteroduplex is introduced into appropriate bacterial cells and clones that include the desired mutation are selected. The resulting altered DNA molecules may be expressed recombinantly in appropriate host cells to produce the modified protein.

Equivalent DNA constructs that include code for additions or substitutions o f amino acid residues or sequences, or deletions of terminal or internal residues or sequences not needed or desired for biological activity, for example, are also encompassed by the invention.

A “host cell” or “recombinant host cell” is a cell that contains a vector, e.g., an expression vector, or a cell that has otherwise been manipulated by recombinant techniques to express a protein of interest. Host organisms include those organisms in which recombinant production of constructs of the invention, for example, preptins, preptin analogs, and/or preptin agonists encoded by the recombinant constructs of the present invention may occur, such as bacteria (for example, E. coli), yeast (for example, Saccharomyces cerevisiae and Pichia pastoris), insect cells, and mammalian cells, including in vitro and in vivo expression. Host organisms thus may include organisms for the construction, propagation, expression or other steps in the production of the compositions provided herein. Hosts include subjects in which immune responses take place, as described herein. Presently preferred host organisms for production of constructs of the invention that produce glycosylated proteins are mammalian cells or other cells systems that permit the expression and recovery of glycosylated proteins. Other cell lines include inbred murine strains and murine cell lines, and human cells and cell lines.

A DNA construct encoding a desired construct to be used in the invention is introduced into a vector, for example, a plasmid, for expression in an appropriate host. In preferred embodiments, the host is a mammalian host, for example, a mammalian cell line. The sequence is preferably codon-optimized for expression in the particular host. Thus, for example, if a construct, for example, is a human preptin or preptin analog or derivative or agonist and is expressed in bacteria, the codons may be optimized for bacterial usage. For small coding regions, the gene can be synthesized as a single oligonucleotide. For larger proteins, splicing of multiple oligonucleotides, mutagenesis, or other techniques known to those in the art may be used. The sequences of nucleotides in plasmids or other vectors that are regulatory regions, such as promoters and operators, are operationally associated with one another for transcription. The sequence of nucleotides encoding a desired protein may also include DNA encoding a secretion signal, whereby the resulting peptide is a precursor protein. The resulting processed protein may be recovered from the periplasmic space or the fermentation medium

In preferred embodiments, the DNA plasmids may also include a transcription terminator sequence. As used herein, a “transcription terminator region” is a sequence that signals transcription termination. The entire transcription terminator may be obtained from a protein-encoding gene, which may be the same or different from an inserted gene or the source of the promoter. Transcription terminators are optional components of the expression systems herein, but are employed in preferred embodiments.

The plasmids or other vectors used herein include a promoter in operative association with the DNA encoding the protein- or polypeptide of interest and are designed for expression of proteins in a suitable host as described above (e.g., bacterial, murine, or human) depending upon the desired use of the plasmid (e.g., gene therapy). Suitable promoters for expression of proteins and polypeptides herein are widely available and are well known in the art. Inducible promoters or constitutive promoters that are linked to regulatory regions are preferred. Such promoters include, for example, but are not limited to, the T7 phage promoter and other T7-like phage promoters, such as the T3, T5 and SP6 promoters, the trp, lpp, and lac promoters, such as the lacUV5, from E. coli; the P10 or polyhedrin gene promoter of baculovirus/insect cell expression systems (see, e.g., U.S. Pat. Nos. 5,243,041, 5,242,687, 5,2661317, 4,745,051, and 5,169,784) and inducible promoters from other eukaryotic expression systems. For expression of the proteins such promoters are inserted in a plasmid in operative linkage with a control region such as the lac operon.

Preferred promoter regions are those that are inducible and functional in mammalian cells, for example: Examples of suitable inducible promoters and promoter regions for bacterial expression include, but are not limited to: the E. coli lac operator responsive to isopropyl β-D-thiogalactopyranoside (IPTG; see Nakamura et al., 1979 Cell 18:1109-1117); the metallothionein promoter metal-regulatory-elements responsive to heavy-metal (e.g., zinc) induction (see e.g., U.S. Pat. No. 4,870,009); the phage T7lac promoter responsive to IPTG (see, e.g., U.S. Pat. No. 4,952,496; and Studier et al., 1990 Meth. Enzymol. 185:60-89) and the TAC promoter. Depending on the expression host system to be used, plasmids may optionally include a selectable marker gene or genes that are functional in the host. Thus, for example, a selectable marker gene includes any gene that confers a phenotype on bacteria that allows transformed bacterial cells to be identified and selectively grown from among a vast majority of untransformed cells. Suitable selectable marker genes for bacterial hosts, for example, include the ampicillin resistance gene (Amp^{r}), tetracycline resistance gene (Tcr) and the kanamycin resistance gene (Kan^{r}). The kanamycin resistance gene is presently preferred for bacterial expression.

In various expression systems, plasmids or other vectors may also include DNA encoding A signal for secretion of the operably linked protein. Secretion signals suitable for use are widely available and are well known in the art. Prokaryotic and eukaryotic secretion signals functional in E. coli may be employed. Depending on the expression systems, presently preferred secretion signals may include, but are not limited to, those encoded by the following E. coli genes: ompA, ompT, ompF, ompC, beta-lactamase, and alkaline phosphatase, and the like (von Heijne, J. Mol. Biol. 184:99-105, 1985). In addition, the bacterial pelB gene secretion signal (Lei et al., J. Bacteriol. 169:4379, 1987), the phoA secretion signal, and the cek2 functional in insect cell may be employed. The most preferred secretion signal for certain expression systems is the E. coli ompA secretion signal. Other prokaryotic and eukaryotic secretion signals known to those of skill in the art may also be employed (see, e.g., von Heijne, J. Mol. Biol. 184:99-105, 1985). Using the methods described herein, one of skill in the art can substitute secretion signals that are functional in either yeast, insect or mammalian cells to secrete proteins from those cells.

Preferred plasmids for transformation of E. coli cells include the pET expression vectors (e.g., pET-11a, pET-12a-c, pET-15b; see U.S. Pat. No. 4,952,496; available from Novagen, Madison, Wis.). Other preferred plasmids include the pKK plasmids, particularly pKK 223-3, which contains the tac promoter (Brosius et al., 1984 Proc. Natl. Acad. Sci. 81:6929; Ausubel et al., Current Protocols in Molecular Biology; U.S. Pat. Nos. 5,122,463, 5,173,403, 5,187,153, 5,204,254, 5,212,058, 5,212,286, 5,215,907, 5,220,013, 5,223,483, and 5,229,279). Plasmid PKK has been modified by replacement of the ampicillin resistance gene with a kanaamycin resistance gene. (Available from Pharmacia; obtained from pUC4K, see, e.g., Vieira et al. (1982 Gene 19:259-268; and U.S. Pat. No. 4,719,179.) Baculovirus vectors, such as pBlueBac (also called pJVETL and derivatives thereof), particularly pBlueBac III (see, e.g., U.S. Pat. Nos. 5,278,050, 5,244,805, 5,243,041, 5,242,687, 5,266,317, 4,745,051, and 5,169,784; available from Invitrogen, San Diego) may also be used for expression of the polypeptides in insect cells. Other plasmids include the pIN-IIIompA plasmids (see U.S. Pat. No. 4,575,013; see also Duffaud et al., Meth. Enz. 153:492-507, 1987), such as pIN-IIIompA2.

In other embodiments, if one or more DNA molecules is replicated in bacterial cells, an example for a host is E. coli. The preferred DNA molecule is such a system also includes a bacterial origin of replication, to ensure the maintenance of the DNA molecule from generation to-generation of the bacteria. In this way, large quantities of the DNA molecule can be produced by replication in bacteria. In such expression systems, preferred bacterial origins of replication include, but are not limited to, the fl-ori and col E1 origins of replication. Preferred hosts for such systems contain chromosomal copies of DNA encoding T7 RNA polymerase operably linked to an inducible promoter, such as the lacUV promoter (see U.S. Pat. No. 4,952,496). Such hosts include,but are not limited to, lysogens E. coli strains HMS174(DE3)pLysS, BL21 (DE3)pLysS, HMS174(DE3) and BL2 1 (DE3). Strain BL21 (DE3) is preferred. The pLys strains provide low levels of T7 lysozyme, a natural inhibitor of T7 RNA polymerase.

The DNA molecules provided may also contain a gene coding for a repressor protein. The repressor protein is capable of repressing the transcription of a promoter that contains sequences of nucleotides to which the repressor protein binds. The promoter can be derepressed by altering the physiological conditions of the cell. For example, the alteration can be accomplished by adding to the growth medium a molecule that inhibits the ability to interact with the operator or with regulatory proteins or other regions of the DNA or by altering the temperature of the growth media. Preferred repressor proteins include, but are not limited to the E. coli lacI repressor responsive to IPTG induction, the temperature sensitive λ cI857 repressor, and the like. The E. coli lacI repressor is preferred.

In general, recombinant constructs of the subject invention will also contain elements necessary for transcription and translation. In particular, such elements are preferred where the recombinant expression construct containing nucleic acid sequences encoding preptin, preptin analog, and/or preptin agonist proteins is intended for expression in a host cell or organism. In certain embodiments of the present invention, cell type preferred or cell type specific expression may be achieved by placing the gene under regulation of a promoter. The choice of the promoter will depend upon the cell type to be transformed and the degree or type of control desired. Promoters can be constitutive or active and may further be cell type specific, tissue specific, individual cell specific, event specific, temporally specific or inducible. Cell-type specific promoters and event type specific promoters are preferred. Examples of constitutive or nonspecific promoters include the SV40 early promoter (U.S. Pat. No. 5,118,627), the SV40 late promoter (U.S. Pat. No. 5,118,627), CMV early gene promoter (U.S. Pat. No. 5,168,062), and adenovirus promoter. In addition to viral promoters, cellular promoters are also amenable within the context of this invention. In particular, cellular promoters for the so-called housekeeping genes are useful. Viral promoters are preferred, because generally they are stronger promoters than cellular promoters. Promoter regions have been identified in the genes of many eukaryotes including higher eukaryotes, such that suitable promoters for use in a particular host can be readily selected by those skilled in the art.

Inducible promoters may also be used. These promoters include MMTV LTR (PCT WO 91/13160), inducible by dexamethasone; metallothionein promoter, inducible by heavy metals; and promoters with cAMP response elements, inducible by cAMP. By using an inducible promoter, the nucleic acid sequence encoding a preptin, a preptin analog, and/or a preptin agonist protein may be delivered to a cell by the subject invention expression construct and will remain quiescent until the addition of the inducer. This allows further control on the timing of production of the gene product.

Event-type specific promoters are active or up-regulated only upon the occurrence of an event, such as tumotigenicity or viral infection. The HIV LTR is a well known example of an event-specific promoter. The promoter is inactive unless the tat gene product is present, which occurs upon viral infection. Some event-type promoters are also tissue-specific.

Additionally, promoters that are coordinately regulated with a particular cellular gene may be used. For example, promoters of genes that are coordinately expressed may be used when expression of a particular construct of the invention, for example, a preptin-, a preptin analog-, and/or a preptin agonist-encoding gene is desired in concert with expression of one or more additional endogenous or exogenously introduced genes. This type of promoter is especially useful when one knows the pattern. of gene expression relevant to induction of an immune response in a particular tissue of the inimune system, so that specific inrnunocompetent cells within that tissue may be activated or otherwise recruited to participate in the immune response.

In addition to the promoter, repressor sequences, negative regulators, or tissue-specific silencers may be inserted to reduce non-specific expression of preptin-, preptin analog- and/or preptin agonist-encoding genes in certain situations, such as, for example, a host that is transiently immunocompromised as part of a therapeutic strategy. Multiple repressor elements may be inserted in the promoter region. Repression of transcription is independent on the orientation of repressor elements or distance from the promoter. One type of repressor sequence is an insulator sequence. Such sequences inhibit transcription (Dunaway et al., 1997 Mol Cell Biol 17: 182-9; Gdula et al., 1996 Proc Natl Acad Sci USA 93:9378-83, Chan et al., 1996 J Virol 70: 5312-28; Scott and Geyer, 1995 EMBO J 14:6258-67; Kalos and Fournier, 1995 Mol Cell Biol 15:198-207; Chung et al., 1993 Cell 74: 505-14) and will silence undesired background transcription.

Repressor elements have also been identified in the promoter regions of the genes for type II (cartilage) collagen, choline acetyltransferase, albumin (Hu et al., 1992 J. Cell Growth Differ. 3(9):577-588), phosphoglycerate kinase (PGK-2) (Misuno et al., 1992 Gene 119(2):293-297), and in the 6-phosphofructo-2-kinase/fructose-2, 6-bisphosphatase gene. (Lemaigre et al., Mol. Cell Biol. 11(2):1099-1106). Furthermore, the negative regulatory element Tse-1 has been identified in a number of liver specific genes, and has been shown to block cAMP response element(CRE)-mediated induction of gene activation in hepatocytes. (Boshart et al., 1990 Cell 61(5):905-916,).

In preferred embodiments, elements that increase the expression of the desired product may be incorporated into the construct. Such elements include internal ribosome binding sites (IRES; Wang and Siddiqui, 1995 Curr. Top. Microbiol. Immunol 203:99; Ehrenfeld and Semler, 1995 Curr. Top. Microbiol. Immunol. 203:65; Rees et al., 1996 Biotechniques 20:102; Sugimoto et al., 1994 Biotechnology 12:694). IRES increase translation efficiency. As well, other sequences may enhance expression. For some genes, sequences especially at the 5′ end inhibit transcription and/or translation. These sequences are usually palindromes that can form hairpin structures. Any such sequences in the nucleic acid to be delivered are generally deleted. Expression levels of the transcript or translated product are assayed to confirm or ascertain which sequences affect expression. Transcript levels may be assayed by any known method, including Northern blot hybridization, RNase probe protection and the like. Protein levels may be assayed by any known method, including ELISA, western blot, immunocytochemistry or other well known techniques.

Other elements may be incorporated into the constructs of the invention, for example, into preptin or preptin analog or agonist protein encoding constructs of the present invention. In preferred embodiments, the construct includes a transcription terminator sequence, including a polyadenylation sequence, splice donor and acceptor sites, and an enhancer. Other elements useful for expression and maintenance of the construct in mammalian cells or other eukaryotic cells may also be incorporated (e.g., origin of replication). Because the constructs are conveniently produced in bacterial cells, elements that are necessary for, or that enhance, propagation in bacteria are incorporated. Such elements include an origin of replication, a selectable marker and the like.

As noted herein, host cells for production or expression of a construct of the invention, for example, can be a higher eukaryotic cell, such as a mammalian cell, or a lower eukaryotic cell, such as a yeast cell, or the host cell can be a prokaryotic cell, such as a bacterial cell. Representative examples of appropriate host cells according to the present invention include, but need not be limited to, bacterial cells, such as E. coli, Streptomyces, Salmonella typhimurium; fungal cells, such as yeast; insect cells, such as Drosophila S2 and Spodoptera Sf9; animal cells, such as CHO, COS or 293 cells; adenoviruses; plant cells, or any suitable cell already adapted to in vitro propagation or so established de novo. The selection of an appropriate host is deemed to be within the scope of those skilled in the art from the teachings herein. Various mammalian cell culture systems can also be employed to express recombinant protein. Examples of mammalian expression systems include the COS-7 lines of monkey kidney fibroblasts, described by Gluzman, 1981 Cell 23:175, and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements. Introduction of the construct into the host cell can be effected by a variety of methods with which those skilled in the art will be familiar, including but not limited to, for example, calcium phosphate transfection, DEAE-Dextran mediated transfection, or electroporation (Davis et al., 1986 Basic Methods in Molecular Biology).

The present invention also relates to vectors, and to constructs prepared from known vectors that include nucleic acids of the present invention, and in particular to “recombinant expression constructs”, including any of various known constructs, including delivery constructs, useful for gene therapy, that include any nucleic acids encoding, for example, preptins, preptin analogs, and preptin agonists according to the invention as provided herein; to host cells which are genetically engineered with vectors and/or other constructs of the invention and to methods of administering expression or other constructs comprising nucleic acid sequences encoding, for example, preptins, preptin analogs, and preptin agonists according to the invention, or fragments or variants thereof, by recombinant techniques.

Various constructs of the invention can be expressed in virtually any host cell, including in vivo host cells in the case of use for gene therapy, under the control of appropriate promoters, depending on the nature of the construct (e.g., type of promoter, as described above), and on the nature of the desired host cell (e.g., whether postmitotic terminally differentiated or actively dividing; e.g., whether the expression construct occurs in host cell as an episome or is integrated into host cell genome). Appropriate cloning and expression vectors for use with prokaryotic and eukaryotic hosts are described, for example, by Sambrook, et al., Molecular Cloning: A Laboratory Manual, Second Edition, Cold Spring Harbor, N.Y., (1989); as noted herein, in particularly preferred embodiments of the invention, recombinant expression is conducted in mammalian cells that have been transfected or transformed with the subject invention recombinant expression construct. See also, for example, Machida, Calif., “Viral Vectors for Gene Therapy: Methods and Protocols”; Wolff, J A, “Gene Therapeutics: Methods and Applications of Direct Gene Transfer” (Birkhauser 1994); Stein, U and Walther, W (eds. P, “Gene Therapy of Cancer: Methods and Protocols” (Humana Press 2000); Robbins, P D (ed.), “Gene Therapy Protocols” (Humana Press 1997); Morgan, J R (ed.), “Gene Therapy Protocols” (Humana Press 2002); Meager, A (ed.), “Gene Therapy Technologies, Applications and Regulations: From Laboratory to Clinic” (John Wiley & Sons Inc. 1999); MacHida, C A and Constant, J G, “Viral Vectors for Gene Therapy: Methods and Protocols” (Humana Press 2002);“New Methods Of Gene Therapy For Genetic Metabolic Diseases NIH Guide,” Volume 22, Number 35, Oct. 1, 1993. See also recent U.S. patents relating to gene therapy, including vaccines, which include U.S. Pat. No. 6,384,210 (“Solvent for biopolymer synthesis, solvent microdroplets and methods of use”); U.S. Pat. No. 6,384,202 (“Cell-specific active compounds regulated by the cell cycle”); U.S. Pat. No. 6,384,018 (“Polynucleotide tuberculosis vaccine”); U.S. Pat. No. 6,383,814 (“Cationic amphiphiles for intracellular delivery of therapeutic molecules”); U.S. Pat. No. 6,383,811 (“Polyampholytes for delivering polyions to a cell”); U.S. Pat. No. 6,383,795 (“Efficient purification of adenovirus”); U.S. Pat. No. 6,383,794 (“Methods of producing high titer recombinant adeno-associated virus”); U.S. Pat. No. 6,383,785 (“Self-enhancing, pharmacologically controllable expression systems”); U.S. Pat. No. 6,383,753. (“Yeast mammalian regulators of cell proliferation”); U.S. Pat. No. 6,383,746 (“Functional promoter for CCR5”); U.S. Pat. No. 6,383,743 (“Method for serial analysis of gene expression”); U.S. Pat. No. 6,383,738 (“Herpes simplex virus ORF P is a repressor of viral protein synthesis”); U.S. Pat. No. 6,383,737 (“Human oxalyl-CoA Decarboxylase”); U.S. Pat. No. 6,383,733 (“Methods of screening for pharmacologically active compounds for the treatment of tumour diseases”); U.S. Pat. No. 6,383,522 (“Toxicity reduced composition containing an anti-neoplastic agent and a shark cartilage extract”); U.S. Pat. No. 6,383,512 (“Vesicular complexes and methods of making and using the same”); U.S. Pat. No. 6,383,481 (“Method for transplantation of hemopoietic stem cells”); U.S. Pat. No. 6,383,478 (“Polymeric encapsulation system promoting angiogenesis”); U.S. Pat. No. 6,383,138 (“Method for transdermal sampling of analytes”); U.S. Pat. No. 6,380,382 (“Gene encoding a protein having diagnostic, preventive, therapeutic, and other uses”); U.S. Pat. No. 6,380,371 (“Endoglycan: a novel protein having selectin ligand and chemokine presentation activity”); U.S. Pat. No. 6,380,369 (“Human DNA mismatch repair proteins”); U.S. Pat. No. 6,380,362 (“Polynucleotides, polypeptides expressed by the polynucleotides and methods for their use”); U.S. Pat. No. 6,380,170 (“Nucleic acid construct for the cell cycle regulated expression of structural genes”); U.S. Pat. No. 6,380,169 (“Metal complex containing oligonucleoside cleavage compounds and therapies”); U.S. Pat. No. 6,379,967 (“Herpesvirus saimiri as viral vector”); U.S. Pat. No. 6,379,966(“Intravascular delivery of non-viral nucleic acid protease proteins, and uses thereof”).

Typically, for example, expression constructs are derived from plasmid vectors. One preferred construct is a modified pNASS vector (Clontech, Palo Alto, Calif.), which has nucleic acid sequences encoding an ampicillin resistance gene, a polyadenylation signal and a T7 promoter site. Other suitable mammalian expression vectors are well known (see, e.g., Ausubel et al., 1995; Sambrook et al., supra; see also, e.g., catalogues from Invitrogen, San Diego, Calif.; Novagen, Madison, Wis.; Pharmacia, Piscataway, N.J.; and others). Presently preferred constructs may be prepared that include a dihydrofolate reductase (DHFR) encoding sequence under suitable regulatory control, for promoting enhanced production levels of preptins, preptin analogs, and preptin agonists, which levels result from gene amplification following application of an appropriate selection agent (e.g., methetrexate).

Generally, recombinant expression vectors will include origins of replication and selectable markers permitting transformation of the host cell, and a promoter derived from a highly-expressed gene to direct transcription of a downstream structural sequence, as described above. The heterologous structural sequence is assembled in appropriate phase with translation initiation and termination sequences. Thus, for example, the preptins, preptin analogs, and preptin agonists according to encoding nucleic acids as provided herein may be included in any one of a variety of expression vector constructs as a recombinant expression construct for expressing a preptin, preptin analog, and/or preptin agonist polypeptide in a host cell. In certain preferred embodiments the constructs are included in formulations that are administered in vivo. Such vectors and constructs include chromosomal, nonchromosomal and synthetic DNA sequences, e.g., derivatives of SV40; bacterial plasmids; phage DNA; yeast plasmids; vectors derived from combinations of plasmids and phage DNA, viral DNA, such as vaccinia, adenovirus, fowl pox virus, and pseudorabies, or replication deficient retroviruses as described below. However, any other vector may be used for preparation of a recombinant expression construct, and in preferred embodiments such a vector will be replicable and viable in the host.

The appropriate DNA sequence(s) may be inserted into a vector, for example, by a variety of procedures. In general, a DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.); Glover (Ed.) (1985 DNA Cloning Vol. I and II, IRL Press, Oxford, UK); Hames and Higgins (Eds.), (1985 Nucleic Acid Hybridization, IRL Press, Oxford, UK); and elsewhere.

The DNA sequence in the expression vector is operatively linked to at least one appropriate expression control sequence(s) (e.g., a constitutive promoter or a regulated promoter) to direct mnRNA synthesis. Representative examples of such expression control sequences include promoters of eukaryotic cells or their viruses, as described above. Promoter regions can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding preptin, preptin analog, and preptin agonist polypeptide is described herein.

Transcription of the DNA encoding proteins and polypeptides included-within the present invention by higher eukaryotes may be increased by inserting an enhancer sequence into the vector. Enhancers are cis-acting elements of DNA, usually about from 10 to 300 bp that act on a promoter to increase its transcription. Examples including the-SV40 enhancer on the late side of the replication origin bp 100 to 270, a cytomegalovirus early promoter enhancer, the polyoma enhancer on the late side of the replication origin, and adenovirus enhancers.

Gene therapy is the use of genetic material to treat disease. It comprises strategies to replace defective genes or add new genes to cells and/or tissues, and is being developed for application in the treatment of cancer, the correction of metabolic disorders and in the field of immunotherapy. Gene therapies of the invention include the use of various constructs of the invention, with or without a separate carrier or delivery vehicle or constructs, for treatment of the diseases, disorders, and/or conditions noted herein. In vivo gene therapy involves the direct injection of genetic material into a patient or animal model of human disease. With tissue-specific in vivo therapies such as those that aim to treat diabetes, localized gene delivery and/or expression/targeting systems are preferred. Diverse gene therapy vectors have been designed to target specific tissues, and procedures have been developed to physically target specific tissues, for example, using catheter-based technologies, all of which are contemplated herein. Ex vivo approaches to gene therapy are also contemplated herein and involve the removal, genetic modification, expansion and re-administration of a patient's own cells. Examples include β-cell transplantation for diabetes treatment or the genetic modification of progenitor cells. Useful gene therapy vectors include adenoviral vectors, lentiviral vectors, Adeno-associated virus (AAV) vectors, Herpes Simplex Virus (Hsv) vectors, and retroviral vectors. Gene therapies may also be carried out using “naked DNA,” lipsome-based delivery, lipid-based delivery (including DNA attached to positively charged lipids), and electroporation. As provided herein, in certain embodiments, including but not limited to gene therapy embodiments, the vector may be a viral vector such as, for example, a retroviral vector. Miller et al., 1989 BioTechniques 7:980; Coffin and Varmus, 1996 Retroviruses, Cold Spring Harbor Laboratory Press, NY. For example, retroviruses from which the retroviral plasmid vectors may be derived include, but are not limited to, Moloney Murine Leukemia Virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus. Suitable promoters for use in viral vectors generally may include, but are not limited to, the retroviral LTR; the SV40 promoter; and the human cytomegalovirus (CMV) promoter described in Miller, et al. 1989 Biotechniques 7:980-990, or any other promoter (e.g., cellular promoters such as eukaryotic cellular promoters including, but not limited to, the histone, pol III, and β-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein, and may be from among either regulated promoters or promoters as described above.

The present invention constructs or compositions comprising one or more polynucleotides encoding same as described herein (for example, to be administered under conditions and for a time sufficient to permit expression of a preptin, preptin analog, and/or preptin agonist protein in a host cell in vivo or in vitro, for gene therapy, for example, among other things), may be formulated into pharmaceutical compositions for administration according to well known methodologies. Pharmaceutical compositions generally comprise one or more recombinant expression constructs, and/or expression products of such constructs, in combination with a pharmaceutically acceptable carrier, excipient or diluent. Such carriers will be nontoxic to recipients at the dosages and concentrations employed. For nucleic acid-based formulations, or for formulations comprising expression products of the subject invention recombinant constructs, about 0.01 μg/kg to about 100 mg/kg body weight will be administered, for example, typically by the intradermal, subcutaneous, intramuscular or intravenous route, or by other routes. A preferred dosage, for example, is about 1 μg/kg to about 1 mg/kg, with about 5 μg/kg to about 200 μg/kg particularly preferred. It will be evident to those skilled in the art that the number and frequency of administration will be dependent upon the response of the host. “Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

Synthesized peptides may be further purified by methods such as affinity column chromatography or- high-pressure liquid chromatography. Standard physicochemical characterization techniques are known in the art, including NMR (¹³C, ¹H, ¹⁹F, or ³¹P) and IR, which can provide confirmatory evidence of the identity and purity of the synthetic products. Amino acid analysis can also be used to confirm the amino acid composition of the peptide. Mass spectroscopy can be used to identify the molecular weight of synthetic products.

Derivatives may be prepared, for example, by fatty acid derivitization, by glycosylation, or by conjugation to monomethoxy polyethyleneglycol (PEG) and/or phospholipids (including PEG-lipid conjugates). Molecular weights of the exemplary PEGs are 2,000 and 5,000, although other PEGs, including but not limited to PEGs ranging from, for example, 600 to 12,000 may also be used. The lipid portion of a derivative may, by way of example, include saturated or unsaturated PEGs, cholesterol, ceramides with short chain (C8), intermediate chain (C 14) and long chain (C20) fatty amides, etc.

Preptins, as well as preptin analogs and agonists, and salts and derivatives thereof, may also be prepared as dimers or larger aggregates for enhanced function and/or longer half-life.

Preptin agonists can be tested in vitro for their abilities to stimulate proliferation of NIH-3T3 cells, or other like cells, or β-cells including for example, INS-IE β-cells. See the specific examples below. In vivo screening may also be carried out by procedures in the art. See, e.g., Burks and White (2001) Diabetes 50 Suppl 1:S140-5; Flier et al. (2001) Proc Natl Acad Sci USA 98(13):7475-80; Guiot et al. (2001) Diabetes 50 Suppl 1:S188; Kjems et al. (2001) Diabetes 50(9):2001-2012; Moore et al. (2001) Diabetes 50(10):2231-6.

Various methods may be used to obtain variants of preptin from any species, using the sequence information provided by the invention. Such methods include but are not limited to, for example, the screening of cDNA libraries, RT-PCR, screening of genomic libraries and computer aided searching of EST, cDNA, and genomic databases. Such methods are well known to those skilled in the art (see, for example, www.MolecularCloning.com; Current Protocols in Molecular Biology, John Wiley and Sons, Inc). For example, screening of genomic or cDNA libraries is commonly performed using oligonucleotide probes and/or primers. Oligonucleotide probes and/or primers based on the sequence of preptin derived from any of the species herein described can be synthesized and used to identify positive clones in either cDNA or genomic DNA libraries from other organisms by means of hybridization or PCR techniques. Positive clones are clones exhibiting sufficient similarity to the sequence of preptin to hybridize with the oligonucleotide probes or primers under hybridization conditions of desired stringency. Probes and/or primers should be at least about 10, preferably at least about 15 and most preferably at least about 20 nucleotides in length. Hybridization and PCR techniques suitable for use with such oligonucleotide probes and/or primers are well known in the art. Positive clones may be analyzed by restriction enzyme digestion, DNA sequencing, or the like. Clones identified, or suspected, to comprise a preptin-encoding nucleotide sequence can be expressed using techniques well known in the art. Preptin from any species can be obtained by expression of a nucleic acid coding sequence in a suitable host cell using techniques known in the art. Suitable host cells include prokaryotic or eukaryotic organisms or cell lines, for example, yeast, E. coli, insect cells and COS1 cells. Preferably, eukaryotic systems are utilised to express preptin. The recombinant expression vectors of the invention can be used to express preptin in a host cell in order to isolate preptin protein. Purified preptin protein of the invention may be prepared by methods well known in the art, generally comprising introducing into a host cell a recombinant nucleic acid encoding preptin, allowing the protein to be expressed in the host cell and isolating and purifying the protein. Preferably, the recombinant nucleic acid is a recombinant expression vector. Proteins can be isolated from a host cell expressing the protein and purified according to standard procedures of the art, including ammonium sulfate precipitation, column chromatography (e.g., ion exchange, gel filtration, affinity chromatography, etc.) electrophoresis, and ultimately, crystallisation (see generally “Enzyme Purification and Related Techniques”, (1971) Methods in Enzymology, 22:233-577 incorporated herein by reference).

The term “β-cell growth” is defined as an increase in the number of β-cells and/or an increase in the β-cell mass.

The term “β-cell mass” refers to β-cell mass and/or weight.

The term “β-cell proliferation” refers to an increase in β-cell number.

Conditions of decreased or compromised β-cells or β-cell mass include those characterized in whole or in part by sub-normal or insufficient β-cells and/or β-cells mass for normal or desired bodily function.

As used herein, the term “subject” refers to but is not limited to a mammal including humans, domesticated animals (or other animals likely to be seen by a veterinarian), as well as commercials animals, including but not limited to, horses, cattle, pigs, sheep, birds, and so on.

The term “treating” is defined as the application or administration of a composition including an effective or other desired amount of one or more preptins, preptin analogs, preptin agonists, and salts and/or derivatives of any of them, to a subject.

Said subject may have, or have been determined to have, for example, a loss of β-cell mass, number or function, a symptom of a loss of β-cell mass, number or function, a disease or disorder secondary to a loss of β-cell mass, number or function, or a predisposition toward a loss of β-cell mass, number or function, with the purpose to cure, alleviate, relieve, remedy, or ameliorate a loss of β-cell mass, number or function, the symptom of a loss of β-cell mass, number or function, the disease or disorder secondary to a loss of β-cell mass, number or function, or the predisposition toward a loss of β-cell mass, number or function.

“An effective amount” refers to an amount of preptin, a preptin analog, or a preptin agonist that confers a therapeutic or other desired effect on the treated subject, for example, an effect on β-cell mass, number or function. A therapeutic or other desired effect may be objective (i.e., measurable by some test or marker) or subjective (i.e., subject gives an indication of or feels an effect). An effective amount of preptin, a preptin analog, or a preptin agonist described above, or a salt or derivative of any of them, may range from about 10 to about 40 μg/Kg body weight to about 200 to about 500 μg/Kg body weight to about 600 to abut 1000 μg/Kg body weight. Effective doses may vary depending on the route of administration, as well as the possibility o f co-usage with other agents such as those useful for, by way of example only, stimulating β-cell proliferation or increasing β-cell mass by, for example, the use of compounds such as glucagon-like peptide-1 (GLP-1), growth hormone (GH), glucose-dependent insulinotropic polypeptide (GIP) hepatocyte growth factor (HGF), PTH-related protein (PTHrP), betacellulin, placental lactogen (PL) and islet neogenesis-associated protein (INGAP).

The “percent identity” of two amino acid sequences can be determined using the algorithm of Karlin and Altschul (1990), Proc. Natl. Acad. Sci. USA 87: 2264-2268, modified as in Karlin and Altschul (1993), Proc. Natl. Acad. Sci. USA 90: 5873-5877. Such an algorithm is incorporated into the NBLAST and XBLAST programs (version 2.0) of Altschul et al. (1990) J. Mol. Biol. 215: 403-10. BLAST protein searches can be performed with the XBLAST program, score=50, wordlength=3 to obtain amino acid sequences homologous to the peptide molecules described herein. Where gaps exist between two sequences, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used.

A “conservative amino acid substitution” is one in which an amino acid residue is replaced with another residue having a chemically similar or derivitized side chain. Families of amino acid residues having similar side chains, for example, have been defined in the art. These families include, for example, amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrdsine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid analogs (e.g., phosphorylated amino acids) are also contemplated in the present invention, as are peptides substituted with non-naturally occurring amino acids, including but not limited to β-amino acids, β amino acids, and γ amino acids.

As used herein the term “isolated” means, in the case of a naturally occurring material, that the material is or has been removed from, or is no longer associated with, its natural or original environment. For example, a naturally occurring protein or polypeptide present in a living animal is not isolated, but the same nucleic acid or polypeptide, separated from some or all of the co-existing materials in the natural system, is isolated. Such proteins or polypeptides could be part of a composition, and still be isolated in that such composition is not part of its natural environment. The term “isolated”, in the case of non-naturally occurring material, such as a recombinantly manufactured protein or polypeptide of the invention, includes material that is substantially or essentially free from components which normally accompany it during manufacture, such as, for example, proteins and peptides that have been purified to a desired degree, preferably, for example, so that they are at least about 80% pure, more preferably at least about 90%, and still more preferably at least about 95% as measured by techniques known in the art.

The term “substantially purified” refers to peptides that are at least about 60% free, preferably at least about 75% free, and most preferably at least about 90% free, or more, from other components with which they may be associated naturally or during manufacture.

As used herein “purified” does not require absolute purity; rather, it is intended as a relative term where the subject protein or other substance is more pure than in its natural environment within a cell or other environment, such as a manufacturing environment. In practice the material has typically, for example, been subjected to fractionation to remove various other components, and the resultant material has substantially retained its desired biological activity or activities.

The term “diabetes mellitus” refers to any disease or symptom wherein a loss of β-cells or β-cell function is involved, and includes any of the diseases or situations described herein.

A β-cell mediated disease is any disease wherein β-cells are involved, in whole or in any part, in the pathology of the disease. β-cell mediated diseases include, for example, type 1 or type 2 diabetes.

As used herein, “preptin(s)”, “a preptin analog(s)”, and “preptin agonist(s)” are defined to include pharmaceutically acceptable salts or derivatives, and does not exclude a mixture of suitable preptins, preptin analogs or preptin agonists.

Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Hydrochloride and acetate salts are preferred.

Also within the scope of this invention is a pharmaceutical composition that contains an effective amount of one or more of a preptin, a preptin analog, or a preptin agonist, or salt and/or derivative of any of them, and a pharmaceutically acceptable carrier.

The term “pharmaceutically acceptable carrier” refers to a carrier (adjuvant or vehicle) that may be administered to a subject, together with an effective amount of one or more of a preptin, a preptin analog, or a preptin agonist, or salt and/or derivative of any of them, and which does not destroy the pharmacological activity thereof and is nontoxic when administered in doses sufficient to deliver said preptin(s), preptin analog (s), and/or preptin agonists.

Pharmaceutically acceptable carriers that may be used in the pharmaceutical compositions described above include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

To practice methods for treating injuries, including internal and external wounds, or methods for treating β-cell loss or loss of function, or methods for increasing or maintaining β-cell mass, number, or function, one or more preptins, preptin analogs, and/or preptin agonists, or salts of derivatives of any of them, can be administered to a subject. The preptin, preptin analog, or the preptin agonist, salt or derivative, can, for example, be administered in a pharmaceutically acceptable carrier such as physiological saline, in combination with other drugs, and/or together with appropriate excipients. It can, for example, be administered by injection (for example, intravenously, intraarterially, subdermally, intraperitoneally, intramuscularly, or subcutaneously), orally, buccally, nasally, transmucosally, topically, in an ophthalmic preparation, by inhalation, by intracranial injection or infusion techniques. The methods herein contemplate administration of an effective amount of compound or compound composition to achieve a desired or stated effect. Lower or higher doses than those described above may be desired or required. Specific dosage and treatment regimens for any particular subject will of course, as is understood in the art, depend upon a variety of factors, including the activity of the specific compound(s) employed, the age, body weight, general health status, sex, diet, time of administration, rate of excretion, drug combination, the severity and course of the disease, condition or symptoms, the subject's disposition to the disease, condition or symptoms, etc., and the judgment of the treating physician.

A pharmaceutical -composition to be orally administered may be in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

A pharmaceutical composition to be administered parenterally may be in any acceptable dosage form and be administered, for example, by any one of the following administration routes, but not limited to subcutaneous, intravenous, intramuscular, intradermal, intrastemal injection or infusion techniques. For example, a sterile injectable composition (e.g., aqueous or oleaginous suspension or other formulation) can be formulated according to techniques known in the art using suitable dispersing or wetting agents (such as, for example, Tween 80) and suspending agents.

Topical administration of a pharmaceutical composition is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, beniyl alcohol and water. The pharmaceutical compositions of this invention may also be topically applied to the lower intestinal tract by rectal suppository formulation or in a suitable enema formulation. Topically applied transdermal or other patches are also included in this invention.

A pharmaceutical composition may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Ointments may be prepared using, for example, either (1) an oleaginous base, such as one consisting of fixed oils or hydrocarbons, such as white petrolatum or mineral oil, or (2) an absorbent base, such as one consisting of an anhydrous substance or substances that can absorb water including, for example, anhydrous lanolin. Customarily, following formation of the base, whether oleaginous or absorbent, the active ingredient, for example one or more preptins, preptin analogs, and/or preptin agonists, is added in an amount affording the desired concentration.

Creams may be, for example, oil/water emulsions. They may comprise, for example, an oil phase (internal phase), typically, for example, fixed oils, hydrocarbons, and the like, such as waxes, petrolatum, mineral oil, and the like, and an aqueous phase (continuous phase) comprising water and any water-soluble substances, such as added salts. The two phases may be stabilized by use of an emulsifying agent, for example, a surface active agent, such as sodium lauryl sulfate; hydrophilic colloids, such as acacia colloidal clays, veegum, and the like. Upon formation of the emulsion, the active ingredient (one or more preptins, preptin analogs, andor preptin agonists) customarily is added in an amount to achieve the desired concentration.

Gels may comprise a base selected from an oleaginous base, water, or an emulsion-suspension base, such as described herein. A gelling agent may be added to the base that forms a matrix in the base, increasing its viscosity. Examples of gelling agents include, for example, hydroxypropyl cellulose, acrylic acid polymers, and the like. Customarily, the active ingredient (one or more preptins, preptin analogs, andor preptin agonists) is added to the formulation at the desired concentration at a point, for example, at a point preceding addition of the gelling agent.

The invention will be further described in the following examples. It should be understood that these examples are for illustrative purposes only and are not to be construed as limiting this invention in any manner.

### EXAMPLE 1

### Stimulation of Proliferation of NIH-3T3 Cells

NIH-3T3 cells may be used for various purposes including research regarding mitogenicity investigations. See, e.g., Buergisser et al. (1990). BiochemBiophys Res Comm 169(3): 832-839; Burgisser et al. (1991). J Biol. Chem 266(2): 1029-33; Yang et al. (1996). Endocrinol 137(7): 2766-2773; Geddes et al. (2001). Prot Engin 14(1): 61-65. NIH-3T3 cells were seeded in 24 well plates at a density of 3×10⁴ cells/well. Cells were grown at 37° C. for 24 hours in high glucose DMEM (supplemented with 30 mM sodium hydrogen carbonate, 2 mM L-glutamine, 10⁵ IU/l penicillin, 0.01% streptomycin sulphate) and 10% fetal calf serum, after which time they were serum starved for 24 hours in high DMEM and 0.2% BSA. Preptin was added to the wells to give final concentrations of 10, 50 100 and 500 nM of preptin/well, and cells were incubated for a further 15 hours before the addition of 1 μCi of ³H-thymidine to each well. After a further 1 hour incubation, medium was aspirated and cells were washed twice with cold PBS, twice with 5% TCA, and twice with 100% ethanol. Plates were then air-dried for 30 minutes before cells were solubilized with 200 μl of 0.2M NaOH. This alkaline cell lysate was then neutralized with 200 μl of 0.2M HCl before 300 μl aliquots were mixed with 2 ml of Starscint scintillation fluid, and counted on a scintillation counter.

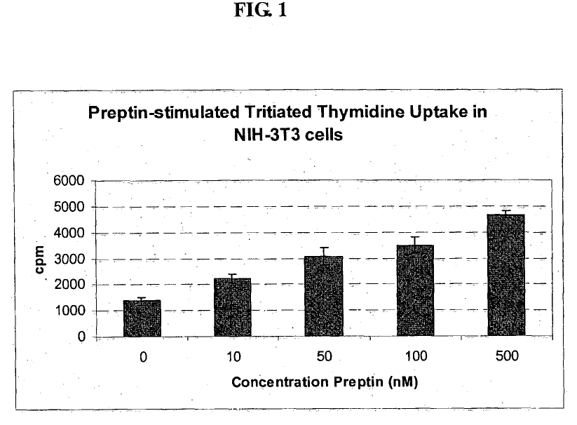

One method of measuring cell proliferation is via the incorporation of labeled nucleotides (e.g., [3H]-thymidine) into- newly synthesized DNA during cell division. For example, one of the most familiar and widely used methods for quantifying cell proliferation is the measurement of tritiated thymidine ([3H]-thymidine) incorporation. Cells incorporate the labeled DNA precursors into newly synthesized DNA, such that the amount of incorporation, measured by liquid scintillation counting, is a relative measure of cellular proliferation. Similarly, in this Example, an increase in counts per minute (cpm) from the scintillation counter indicates an increase in ³H-thymidine uptake, and consequently in cell proliferation. Cell proliferation results are shown in FIG. 1.

### EXAMPLE 2

### Stimulation of Proliferation of INS-1E β-CELLS

INS-1E cells were seeded in 24 well plates at a density of 2×10⁵ cells/well. Cells were grown at 37° C. for 24 hours in modified RPMI-1640 (GIBCO R-1383 Lot108H83032; supplemented with 23.8 mM sodium hydrogen carbonate, 2 mM L-glutamine, 10 mM HEPES, 50 μM β-Mercaptoethanol, 1 mM Sodium Pyruvate, 10⁵ IU/l penicillin, 0.01% streptomycin sulphate) and 10% fetal calf serum, after which time they were serum starved for 24 hours in modified RPMI-1640. The serum free medium was then replaced with 500 μl of RPMI-1640 containing the following:

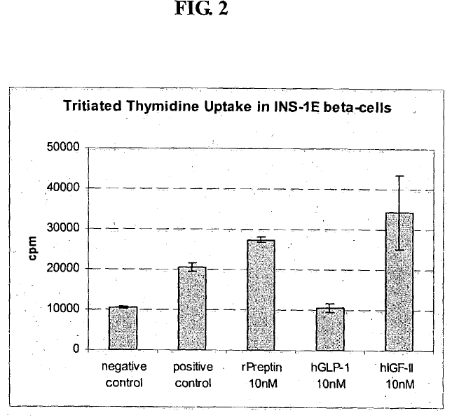

0% fetal calf serum (negative control), 10% fetal calf serum (positive control), 10 nM of rat preptin (rPreptin 10 nM), 10 nM of human GLP-1 (hGLP-1 10 nM), 10 nM of human IGF-II (hIGF-II 10 nM), and cells were incubated for a further 18 hours before the addition of 5 μCi of ³H-thymidine to each well. After a further 6 hours incubation, medium was aspirated and cells were washed twice with cold PBS, once with cold 5% TCA, and twice with cold 100% ethanol. Plates were then air-dried for 30 minutes before cells were solubilized with 400 μl of 0.5M NaOH. This alkaline c ell lysate was then neutralized with 400 μl of 0.5M HCl before 500 μl aliquots were mixed with 2 ml of Starscint scintillation fluid, and counted on a scintillation counter. An increase in counts per minute (cpm) from the scintillation counter indicates an increase in ³H-thymidine uptake, and consequently in cell proliferation. Cell proliferation results are shown in FIG. 2.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily, apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as -defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also understood that the terms “comprising” or “comprises” also refer to the terms “including” or “includes”. It is also to be understood that as used herein and in the appended claims, the singular forms “a,” “an,” and “the” include plural reference unless the context clearly dictates otherwise, the term“X and/or Y” means “X” or “Y” or both “X” and “Y”, and the letter “s” following a noun designates both the plural and singular forms of that noun. In addition, where features or aspects of the invention are described in terms of Markush groups, those skilled in the art will recognize that the invention is also thereby described in terms of any individual member or subgroup of members of the Markush group.

Other embodiments are within the following claims. Under no circumstances may the patent be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A method for treating a condition characterized by or involving, or that may be relieved in any measure by ameliorating, decreased β-cell mass and/or decreased β-cell number in a subject, comprising administering to said subject an effective amount of one or more compounds selected from the group consisting of preptins, preptin analogs, preptin agonists, salts thereof, and derivatives thereof.

2. The method of claim 1, wherein said preptin is selected from the group consisting of a human preptin comprising the amino acid sequence Asp Val Ser Thr Pro Pro Thr Val Leu Pro Asp Asn Phe Pro Arg Tyr Pro Val Gly Lys Phe Phe Gln Tyr Asp Thr Trp Lys Gln Ser Thr Gln Arg Leu (SEQ ID NO: 1), a rat preptin comprising the amino acid sequence Asp Val Ser Thr Ser Gln Ala Val Leu Pro Asp Asp Phe Pro Arg Tyr Pro Val Gly Lys Phe Phe Lys Phe Asp Thr Trp Arg Gln Ser Ala Gly Arg Leu (SEQ ID NO: 2), and a mouse preptin comprising the amino acid sequence Asp Val Ser Thr Ser Gln Ala Val Leu Pro Asp Asp Phe Pro Arg Tyr Pro Val Gly Lys Phe Phe Gln Tyr Asp Thr Trp Arg Gln Ser Ala Gly Arg Leu (SEQ ID NO: 3).

3. The method of claim 1, wherein the preptin agonist comprises a fragment or the entirety of an amino acid sequence of SEQ ID NO: 1, 2, or 3.

4. The method of claim 3, wherein the fragment comprises residues 17-34 of SEQ ID NO: 1, 2, or 3.

5. The method of claim 1, wherein the preptin agonist comprises an amino acid sequence that is at least about 60% identical to SEQ ID NO: 1, 2, or 3.

6. The method of claim 5, wherein the preptin agonist comprises an amino acid sequence that is at least about 80% identical to SEQ ID NO: 1, 2, or 3.

7. The method of claim 5, wherein the preptin agonist comprises an, amino acid sequence that is at least about 90% identical to SEQ ID NO: 1, 2, or 3.

8. The method of claim 5, wherein the preptin agonist comprises an amino acid sequence that is at least about 95% identical to SEQ ID NO: 1, 2, or 3.

9. The method of claim 1, wherein the preptin agonist comprises SEQ ID NO: 1, 2, or 3 with up to about 14 conservative or other amino acid substitutions.

10. The method of claim 9, wherein the preptin agonist comprises SEQ ID NO: 1, 2, or 3 with up to about 10 to 13 conservative or other amino acid substitutions.

11. The method of claim 9, wherein the preptin agonist comprises SEQ ID NO: 1, 2, or 3 with up to about 6 to 9 conservative or other amino acid substitutions.

12. The method of claim 9, wherein the preptin agonist comprises SEQ ID NO: 1, 2, or 3 with up to about 2 to 5 conservative or other amino acid substitutions.

13. A method for increasing or maintaining β-cell mass and/or β-cell number, comprising administering to a subject in need thereof an effective amount of one or more compounds selected from the group consisting of preptins, preptin analogs, preptin agonists, salts thereof, and derivatives thereof.

14. The method of claim 13, wherein said preptin is selected from the group consisting of a human preptin comprising the amino acid sequence Asp Val Ser Thr Pro Pro Thr Val Leu Pro Asp Asn Phe Pro Arg Tyr Pro Val Gly Lys Phe Phe Gln Tyr Asp Thr Trp Lys Gln Ser Thr Gln Arg Leu (SEQ ID NO: 1), a rat preptin comprising the amino acid sequence Asp Val Ser Thr Ser Gln Ala Val Leu Pro Asp Asp Phe Pro Arg Tyr Pro Val Gly Lys Phe Phe Lys Phe Asp Thr Trp Arg Gln Ser Ala Gly Arg Leu (SEQ ID NO: 2), and a mouse preptin comprising the amino acid sequence Asp Val Ser Thr Ser Gln Ala Val Leu Pro Asp Asp Phe Pro Arg Tyr Pro Val Gly Lys Phe Phe Gln Tyr Asp Thr Trp Arg Gln Ser Ala Gly Arg Leu (SEQ ID NO: 3).

15. The method of claim 13, wherein the preptin agonist comprises a fragment or the entirety of the amino acid sequence of SEQ ID NO: 1, 2, or 3.

16. The method of claim 15, wherein the fragment comprises residues 17-34 of SEQ ID NO: 1, 2, or 3.

17. The method of claim 13, wherein the preptin agonist comprises a peptide selected from the group consisting of (1) an amino acid sequence that is at least about 60% identical to SEQ ID NO: 1, 2, or 3, (2) an amino acid sequence that is at least about 80% identical to SEQ ID NO: 1, 2, or 3, (3) an amino acid sequence that is at least about 90% identical to SEQ ID NO: 1, 2, or 3, and (4) an amino acid sequence that is at least 95% identical to SEQ ID NO: 1, 2, or 3.

18. The method of claim 13, wherein the preptin agonist is selected from the group consisting of a peptide comprising (1) SEQ ID NO: 1, 2, or 3 with up to about 14 conservative or other amino acid substitutions, (2) SEQ ID NO: 1, 2, or 3 with up to about 10 to 13 conservative amino or other acid substitutions, (3) SEQ ID NO: 1, 2, or 3 with up to about 6 to 9 conservative or other amino acid substitutions, (4) SEQ ID NO: 1, 2, or 3 with up to about 2 to 5 conservative or other amino acid substitutions.

19. A method for stimulating growth in β-cell proliferation and/or increased β-cell mass, comprising administering to a subject in need thereof an effective amount of one or more compounds selected from the group consisting of preptins, preptin analogs, preptin agonists, salts thereof, and derivatives thereof.

20. The method of claim 19, wherein said preptin is selected from the group consisting of a human preptin comprising the amino acid sequence Asp Val Ser Thr Pro Pro Thr Val Leu Pro Asp Asn Phe Pro Arg Tyr Pro Val Gly Lys Phe Phe Gln Tyr Asp Thr Trp Lys Gln Ser Thr Gln Arg Leu, a rat preptin comprising the amino acid sequence Asp Val Ser Thr Ser Gln Ala Val Leu Pro Asp Asp Phe Pro Arg Tyr Pro Val Gly Lys Phe Phe Lys Phe Asp Thr Trp Arg Gln Ser Ala Gly Arg Leu, and a mouse preptin comprising the amino acid sequence Asp Val Ser Thr Ser Gln Ala Val Leu Pro Asp Asp Phe Pro Arg Tyr Pro Val Gly Lys Phe Phe Gln Tyr Asp Thr Trp Arg Gln Ser Ala Gly Arg Leu.

21. The method of claim 19, wherein the preptin agonist comprises a fragment or the entirety of an amino acid sequence of SEQ ID NO: 1, 2, or 3.

22. The method of claim 21, wherein the fragment comprises amino acid residues 17-34 of SEQ ID NO: 1, 2, or 3.

23. The method of claim 19, wherein the preptin agonist comprises a peptide selected from the group consisting of (1) an amino acid sequence that is at least about 60% identical to SEQ ID NO: 1, 2, or 3, (2) an amino acid sequence that is at least about 80% identical to SEQ ID NO: 1, 2, or 3, (3) an amino acid sequence that is at least about 90% identical to SEQ ID NO: 1, 2, or 3, and (4) an amino acid sequence that is at least 95% identical to SEQ ID NO: 1, 2, or 3.

24. The method of claim 19, wherein the preptin agonist is selected from the group consisting of a peptide comprising (1) SEQ ID NO: 1, 2, or 3 with up to about 14 conservative or other amino acid substitutions, (2) SEQ ID NO: 1, 2, or 3 with up to about 10 to 13 conservative amino or other acid substitutions, (3) SEQ ID NO: 1, 2, or 3 with up to about 6 to 9 conservative or other amino acid substitutions, (4) SEQ ID NO: 1, 2, or 3 with up to about 2 to 5 conservative or other amino acid substitutions.

25. A method of treating a mediated disease, disorder of condition mediated in whole or in part by β-cells or β-cell dysfunction in a subject, comprising administering to the subject an effective amount of one or more compounds selected from the group consisting of preptins, preptin analogs, preptin agonists, salts thereof, and derivatives thereof.

26. The method of claim 25, wherein the said preptin is selected from the group consisting of a human preptin comprising the amino acid sequence Asp Val Ser Thr Pro Pro Thr Val Leu Pro Asp Asn Phe Pro Arg Tyr Pro Val Gly Lys Phe Phe Gln Tyr Asp Thr Trp Lys Gln Ser Thr Gln Arg Leu, a rat preptin comprising the amino acid sequence Asp Val Ser Thr Ser Gln Ala Val Leu Pro Asp Asp Phe Pro Arg Tyr Pro Val Gly Lys Phe Phe Lys Phe Asp Thr Trp Arg Gln Ser Ala Gly Arg Leu, and a mouse preptin comprising the amino acid sequence Asp Val Ser Thr Ser Gln Ala Val Leu Pro Asp Asp Phe Pro Arg Tyr Pro Val Gly Lys Phe Phe Gln Tyr Asp Thr Trp Arg Gln Ser Ala Gly Arg Leu.

27. The method of claim 25, wherein the preptin agonist comprises a fragment or the entirety of the amino acid sequence of SEQ ID NO: 1, 2, or 3.

28. The method of claim 25, wherein the disease is type 1 diabetes or type 2 diabetes.

29. A method of increasing insulin secretion in a subject comprising administering to the subject an effective amount of preptin, a preptin analog, and/or a preptin agonist.

30. The method of claim 29, wherein said preptin is selected from the group consisting of a human preptin comprising the amino acid sequence Asp Val Ser Thr Pro Pro Thr Val Leu Pro Asp Asn Phe Pro Arg Tyr Pro Val Gly Lys Phe Phe Gln Tyr Asp Thr Trp Lys Gln Ser Thr Gln Arg Leu, a rat preptin comprising the amino acid sequence Asp Val Ser Thr Ser Gln Ala Val Leu Pro Asp Asp Phe Pro Arg Tyr Pro Val Gly Lys Phe Phe Lys Phe Asp Thr Trp Arg Gln Ser Ala Gly Arg Leu, and a mouse preptin comprising the amino acid sequence Asp Val Ser Thr Ser Gln Ala Val Leu Pro Asp Asp Phe Pro Arg Tyr Pro Val Gly Lys Phe Phe Gln Tyr Asp Thr Trp Arg Gln Ser Ala Gly Arg Leu.

31. The method of claim 29, wherein the preptin agonist comprises a fragment or the entirety of the amino acid sequence of SEQ ID NO: 1, 2, or 3.

32. An article of manufacture comprising:
a vessel containing an amount of one or more compounds selected from the group consisting of preptins, preptin analogs, preptin agonists, salts thereof, and derivatives thereof in an amount effective to treat or ameliorate a disease, condition or disorder, or one more symptoms thereof; and
instructions for use of the contents of the vessel for the treatment or amelioration of a disease, condition or disorder involving an injury, a wound, decreased β-cell mass, decreased β-cell number, or decreased β-cell function, or one more symptoms thereof, comprising administration to a subject.

33. An article of manufacture comprising:
packaging material; and
contained within the packaging material, one or more compounds selected from the group consisting of preptins, preptin analogs, preptin agonists, salts thereof, and derivatives thereof, in an amount effective to treat or ameliorate a disease, condition or disorder, or one more symptoms thereof;
wherein the packaging material describes or refers to the use of said one or more compounds selected from the group consisting of preptins, preptin analogs, preptin agonists, salts thereof, and derivatives thereof, for treating an injury, a wound, or a condition mediated in whole or in part by β-cell loss or β-cell dysfunction in a subject.

34. The use of one or more compounds selected from the group consisting of preptins, preptin analogs, preptin agonists, salts thereof, and derivatives thereof, in the manufacture of a medicament for the treatment of a subject of any one or more of the following:
i) an internal injury;
ii) an external injury;
iii) an internal wound;
iv) an external wound,
v) a condition characterized in whole or in part by decreased β-cell mass
vi) a condition characterized in whole or in part by decreased β-cell number,
vii) increasing or maintaining β-cell mass;
viii) increasing or maintaining β-cell number,
ix) stimulating β-cell proliferation via cell differentiation or neogenesis,
x) increasing β-cell mass via cell differentiation or neogenesis,
xi) β-cell mediated disease;
xii) a condition characterized in whole or in part by undesirably low insulin secretion; and
xiii) a condition characterized in whole or in part by insulin resistance;
xiv) a condition characterized in whole or in part by hyperglycemia; and,
xv) a condition characterized in whole or in part by postprandial hyperglycemia.

35. The use of claim 34, wherein said preptin is a human, a rat, or a mouse preptin.

36. The use of claim 34, wherein the preptin agonist comprises a fragment or the entirety of the amino acid sequence of SEQ ID NO: 1, 2, or 3.

37. The use of claim 36, wherein the fragment comprises amino acid residues 17-34 of SEQ ID NO: 1, 2, or 3.

38. The use of claim 34, wherein the preptin agonist comprises a peptide selected from the group consisting of (1) an amino acid sequence that is at least about 60% identical to SEQ ID NO: 1, 2, or 3, (2) an amino acid sequence that is at least about 80% identical to SEQ ID NO: 1, 2, or 3, (3) an amino acid sequence that is at least about 90% identical to SEQ ID NO: 1, 2, or 3, and (4) an amino acid sequence that is at least 95% identical to SEQ ID NO: 1, 2, or 3.

39. The use of claim 34, wherein the preptin agonist is selected from the group consisting of a peptide comprising (1) SEQ ID NO: 1, 2, or 3 with up to about 14 conservative or other amino acid substitutions, (2) SEQ ID NO: 1, 2, or 3 with up to about 10 to 13 conservative amino or other acid substitutions, (3) SEQ ID NO: 1, 2, or 3 with up to about 6 to 9 conservative or other amino acid substitutions, (4) SEQ ID NO: 1, 2, or 3 with up to about 2 to 5 conservative or other amino acid substitutions.

40. The use claim 34 wherein the β-cell mediated disease is either type 1 diabetes or type 2 diabetes.

41. A dosage unit useful or suitable for use in a method of any one of claims 1 to 40 comprising one or more compounds selected from the group consisting of preptins, preptin analogs, preptin agonists, salts thereof, and derivatives thereof, formulated to be administered or self administered with any one or more of a pharmaceutically acceptable encapsulation or matrix, a pharmaceutically acceptable carrier, a pharmaceutically acceptable suitable co-active, a pharmaceutically acceptable buffer, a pharmaceutically acceptable salt, a pharmaceutically acceptable tonicity agent, and/or a pharmaceutically acceptable diluent.

42. The dosage unit of claim 41 wherein the amount of said one or more compounds selected from the group consisting of preptins, preptin analogs, preptin agonists, salts thereof, and derivatives thereof, is within the range from about 10 to about 40 μg/Kg body weight of a subject to about 200 to about 500 μg/Kg body weight of a subject to about 600 to abut 1000 μg/Kg body weight of a subject.

43. A method for treating an injury or wound in or on a subject, which comprises applying to said injury or wound a composition comprising an effective amount of one or more compounds selected from the group consisting of preptins, preptin analogs, preptin agonists, salts thereof, and derivatives thereof.

44. The method of claim 43, wherein said subject is a human.

45. The method of claim 43, wherein said subject is not a human.

46. The method of any of claims 43, 44, or 45, wherein said wound is one in which the skin or another external surface is torn, pierced, cut, or otherwise broken.

47. The method of any of claims 43, 44, or 45, wherein said wound is one that penetrates the flesh but does not substantially damage underlying bones or vital organs.

48. The method of any of claims 43, 44, or 45, wherein said wound is a skin surface injury.

49. The method of any of claims 43, 44, or 45, wherein said wound is internal bleeding.

50. The method of any of claims 43, 44, or 45, wherein said composition is an ointment, a cream, or a gel.

51. The method of any of claims 43, 44, or 45, wherein said wound is selected from the group consisting of chemical burns, thermal burns, skin graft donor sites, skin graft transplant sites, cutaneous ulcers, surgical wounds, wound dehiscence, corneal trauma, corneal transplant sites, tooth extraction sites, oral surgery wounds, disruption of a mucous membrane, a disruption of the skin, and a disruption of the connective tissue.

52. The method of claim 51 wherein said cutaneous ulcer is selected from the group consisting of decubitus ulcers, diabetic ulcers, vascular stasis ulcers, and necrobiosis lipoidicum ulcers.

53. The method of claim 51 wherein said mucous membrane disruption is selected from the group consisting of a disruption of a mucous membrane within the gastrointestinal tract, and a disruption of a mucous membrane within the bladder.

54. The method of claim 53 wherein said disruption of a mucous membrane within the gastrointestinal tract comprises ulcerative colitis.

55. The method of claim 53 wherein said disruption of a mucous membrane within the gastrointestinal tract comprises Crohn's Disease.

56. The method of claim 51 wherein said disruption of the skin comprises an abrasion.

57. The method of claim 51 wherein said disruption of the connective tissue comprises an abrasion.

58. A method for enhancing wound healing, which comprises topically applying to such wound an effective amount of one or more compounds selected from the group consisting of selected from the group consisting of preptins, preptin analogs, preptin agonists, salts thereof, and derivatives thereof.

59. A method for treating a condition in a subject by the application or administration of a compound that promotes the proliferation of mesenchymal-derived cells and/or cell mass, the improvement comprising administering to said subject an effective amount of one or more compounds selected from the group consisting of preptins, preptin analogs, preptin agonists, salts thereof, and derivatives thereof.

60. The method of claim 59, wherein the mesenchymal-derived cells comprise fibroblasts.

61. Administration of a composition comprising an effective amount of one or more compounds selected from the group consisting of preptins, preptin analogs, preptin agonists, salts thereof, and derivatives thereof, for the treatment and/or prevention of peripheral nervous system injury, decreasing cell death of motor neurons, increasing muscular end plates, promoting the functional recovery of damaged sciatic nerves, preventing peripheral motor paralysis during or as a result of chemotherapy, treatment and/or prevention of Alzheimer's disease, treatment and/or prevention of apoplexy, treatment and/or prevention of amyotrophic lateral sclerosis, treatment and/or prevention of Parkinson's disease, treatment and/or prevention of muscular dystrophy, treatment and/or prevention of diabetic neuropathy, improvement of myocardial function, treatment and/or prevention of myocardiopathies including myocarditis and myocardial infarction, treatment and/or prevention of cardiac disease and acute attack, and treatment and/or prevention of acute renal insufficiency caused by ischemia.

62. A method for promoting the growth of tissues in a subject by the application or administration of a compound that promotes such growth, which comprises applying or administering to said subject an effective amount of one or more compounds selected from the group consisting of preptins, preptin analogs, preptin agonists, salts thereof, and derivatives thereof.

63. The method of claim 62 wherein the tissue is selected from the group consisting of connective tissue and epithelial tissue.

64. A method for improving the immune function in a subject by the application or administration of a compound to improve immune function, which comprises applying or administering to said subject an effective amount of one or more compounds selected from the group consisting of preptins, preptin analogs, preptin agonists, salts thereof, and derivatives thereof.

65. The method of claim 64 wherein the improvement of said immune function comprises an improvement in lymphocyte proliferation.
